SEQUENCE LISTING (6 sequences)

SEQ ID NO: 1 - protein, 513 aa, Canis lupus
MALIRKRRAAPQDIYPACKVSNTCPADILNKMEQNTLADKILKYGSAGVFLGGLGISTGKGVGGRTGYIPLGGTESGVGVGTRVTTIRPTVPISSVGSPDFIPVDAVDPLGPAVIPPERFPIAVEDPFTLPPPRFPTAVEEDVIELQPIPGPSSEIPLAGPKITTDAQPAILEVIPETRPPKVITRHQYSNPAFEVSITSNSGAGESSASDHVLVEGFSGGHSIGEHIPLQDLAPSRPSFSETIEDETAFSSSTPKQGSRSERPKSYYNRRRYQQVQVTDPVFISRPRSLVTFDNPAFDESVDLIFERDVAEITAAPHADFTDITKLTKPAYHRGPSGHVRVSRLGHRANIKTRSGLTIGPQSHFYYDVSSIDPAESFELQALGNVSSAEQTGEAVISSGTGDFEIISLEDSILESYNDEDLIDVFEDVARDLHLLVGERRQQPIQVQRYIKPFSFVNEGVHIIHPGSESDFWLPPVTPDSTPAIVIDILDSSADYYLHPSLIKKRKRKHFFF

SEQ ID NO: 2 - DNA, 1542 bp, Canis lupus
atggcattgatcaggaaaagacgcgcagcccctcaagatatataccctgcttgtaaagtgtccaacacttgccccgctgatattttgaataaaatggagcaaaatacgcttgcagataaaatcctcaaatatggtagtgctggtgtttttttggggggtctaggaatatcaacaggcaaaggggtaggggggcgcacaggttacattcctttgggaggaacagaaagtggagtgggtgtaggcacaagggtcacaacaataagacctactgtcccaataagcagtgtggggtctcctgactttattcctgtagatgcagtagaccctctagggcctgcagtcatacccccagaaagatttcctatagcagtagaggatccttttactctacctccaccacgtttcccaactgcagtagaagaagatgtaattgagctgcagcctattccaggcccctcatctgaaatcccactcgctggccctaagattaccactgatgctcagcctgcaatattggaagtcataccagagactaggcctcctaaagtaatcactaggcatcagtacagtaaccctgcatttgaggtgtccattacttctaactctggtgcaggggagtcttctgcttctgaccatgtgctggtggaaggtttttctggtggccactcaattggtgaacacattccattgcaagaccttgcacccagcaggccttcattctctgagaccatagaagatgaaactgcttttagcagcagtactcctaaacaaggctctagatctgaaaggcctaaaagttactataataggcgaagatatcagcaagtacaagttactgaccctgtgtttatttcaagaccacggtcacttgtcacgtttgataacccagcctttgatgaatctgttgacctgatatttgaaagagatgttgcagaaataactgcagcacctcatgcagactttacagatatcacaaagctcacaaagcctgcatatcacagaggcccatctggccatgtccgtgtcagtaggcttggacatagagctaatataaaaactagaagtggtcttacaatagggccacaaagccatttttactacgatgtcagcagcattgaccctgcagaatcttttgagctgcaggcacttggcaatgtatccagtgctgaacaaacaggagaagcagtaatctcctctggcacaggagactttgaaattataagccttgaagacagtattttggaatcctacaatgatgaggatttaatagacgtgtttgaggatgtagctagagatttgcatttattagttggagaaagaaggcagcaaccgatccaagttcaacgttacataaagcctttttcttttgttaatgagggagtacacataattcacccaggatctgagtcagatttttggctgcctcctgtaacgcctgacagcacacctgcaatagtgattgacattttggactcctctgcagattactatctgcatccaagtttaataaaaaaacgcaaacgcaaacatttttttttttaa

SEQ ID NO: 3 - protein, 473 aa, Homo sapiens
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 4 - DNA, 1422 bp, Homo sapiens
atgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctag

SEQ ID NO: 5 - protein, 462 aa, Homo sapiens
MVSHRAARRKRASVTDLYKTCKQSGTCPPDVVPKVEGTTLADKILQWSSLGIFLGGLGIGTGSGTGGRTGYIPLGGRSNTVVDVGPTRPPVVIEPVGPTDPSIVTLIEDSSVVTSGAPRPTFTGTSGFDITSAGTTTPAVLDITPSSTSVSISTTNFTNPAFSDPSIIEVPQTGEVAGNVFVGTPTSGTHGYEEIPLQTFASSGTGEEPISSTPLPTVRRVAGPRLYSRAYQQVSVANPEFLTRPSSLITYDNPAFEPVDTTLTFDPRSDVPDSDFMDIIRLHRPALTSRRGTVRFSRLGQRATMFTRSGTQIGARVHFYHDISPIAPSPEYIELQPLVSATEDNDLFDIYADDMDPAVPVPSRSTTSFAFFKYSPTISSASSYSNVTVPLTSSWDVPVYTGPDITLPSTTSVWPIVSPTAPASTQYIGIHGTHYYLWPLYYFIPKKRKRVPYFFADGFVAA

SEQ ID NO: 6 - DNA, 1389 bp, Homo sapiens
atggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctag

(19) United States
(12) Patent Application Publication
(10) Pub. No.: US 2010/0221280 A1
(43) Pub. Date: Sep. 2, 2010

(54) PLANT-PRODUCED COMPOSITIONS FOR TREATING PAPILLOMAVIRUS INFECTION AND RELATED METHODS

(75) Inventors: Shin-je Ghim, Louisville, KY (US); A. Bennett Jenson, Louisville, KY (US); Amanda B. Lasnik, Louisville, KY (US); Donald M. Miller, Louisville, KY (US); Kenneth Palmer, Louisville, KY (US); Mark L. Smith, Davis, CA (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 12/764,183

(22) Filed: Apr. 21, 2010

Related U.S. Application Data
(63) Continuation of application No. 12/028,721, filed on Feb. 8, 2008, now abandoned.
Application No. 12/028,721 is a continuation-in-part of application No. 11/950,366, filed on Dec. 4, 2007, now abandoned.
Application No. 11/950,366 is a continuation of application No. 11/107,575, filed on Apr. 15, 2005, now abandoned.
Application No. 12/028,721 is a continuation-in-part of application No. 11/518,549, filed on Sep. 8, 2006, now abandoned.
(60) Provisional application No. 60/888,873, filed on Feb. 8, 2007.
(60) Provisional application No. 60/563,071, filed on Apr. 15, 2004.
(60) Provisional application No. 60/715,703, filed on Sep. 8, 2005.

(51) Int. Cl. A61K 39/00; C07K 14/025; A61K 39/295; A61K 39/12; A61P 31/12; C12P 21/06

(52) U.S. Cl. 424/192.1; 530/350; 424/202.1; 424/204.1; 435/69.1; 435/69.7

(57) ABSTRACT

Compositions for treating papillomavirus (PV) infection in a subject include a PV L2 polypeptide produced from a eukaryotic expression system.

### RELATED APPLICATIONS

This application is a continuation of U.S. patent application Ser. No. 12/028,721 filed Feb. 8, 2008, which claims priority from U.S. Provisional Application Ser. No. 60/888,873 filed Feb. 8, 2007; this application is a continuation-in-part of U.S. patent application Ser. No. 11/950,366 filed on Dec. 4, 2007, which is a continuation of U.S. patent application Ser. No. 11/107,575 filed Apr. 15, 2005, which claims priority from U.S. Provisional Application Ser. No. 60/563,071 filed Apr. 15, 2004; and this application is a continuation-in-part of U.S. patent application Ser. No. 11/518,549 filed on Sep. 8, 2006 and claiming priority from U.S. Provisional Application Ser. No. 60/715,703. The entire disclosures contained in U.S. application Ser. Nos. 12/028,721; 60/888,873; 60/715,703; 60/563,071; 11/518,549; 11/107,575; and 11/950,366 are incorporated herein by this reference.

### GOVERNMENT INTEREST

Certain subject matter described herein was made with government support under Grant Number 1R01DP000214 awarded by the Centers for Disease Control and Prevention, and Grant Number 70NANB2H3048 awarded by the National Institutes for Standards and Technologies. The government has certain rights in the described subject matter.

### TECHNICAL FIELD

The presently-disclosed subject matter relates to the treatment of papillomavirus infections.

### INTRODUCTION AND GENERAL CONSIDERATIONS

Papillomaviruses are species-specific, anatomic-site restricted small DNA tumor viruses that cause a variety of pathologies of differing degrees of severity. Certain types of papillomaviruses (PVs) can cause papillomas or warts. These warts can occur in a variety of locations on an infected subject. Warts caused by PV are sometimes found in the genital region of an infected subject. In some cases, these warts can infect babies born to infected mothers. In such a situation, the child can develop recurrent respiratory papillomatosis (RRP), where papillomas develop in the respiratory tract. These respiratory papillomas can be deadly in pediatric RRP due to the small size of the upper airway in children. Papillomaviruses (PVs) are also associated with up to 99% of cervical cancers. Through use of cervical screening tests, the incidence of invasive cervical cancers in developed countries has decreased, but still occurs in a number of women in developed countries. In developing and underdeveloped countries invasive cervical cancer is an even greater threat.

Human papillomaviruses (HPVs) associated with warts include HPV-6 and HPV-11. HPVs implicated in the etiology of cervical cancer include: HPV-16, HPV-18, HPV-31; HPV-33; HPV-35; HPV-39; HPV-45, HPV-52, HPV-58, and HPV-68. Research in the past decade has generated a wealth of knowledge on the correlates of protection against papillomavirus infection. Investigators have attempted to develop compositions to prevent infection with some of the HPV-types known to cause cervical cancer, anogenital cancer, head and neck cancers, other mucosal cancer, and genital warts.

HPVs have two outer coat proteins, the major capsid protein (L1; 95% of coat protein) and the minor capsid protein (L2; 5% of coat protein). A composition effective against certain HPV-types has been produced using virus-like particles (VLPs) of the L1 major capsid protein. Preclinical studies were conducted in a canine oral papillomavirus (COPV) system, which is established as the model-of-choice for use in preclinical studies in the field. Preclinical studies were 100% successful, and clinical studies in humans were 100% successful as well, further establishing the efficacy of the L1-VLP composition. Uninfected women who were vaccinated with the VLPs comprising the L1 major capsid protein of HPV-16 were protected against acquisition of chronic HPV-16 infection and development of cervical intraepithelial neoplasia (CIN), e.g., CIN-II, which is the premalignant lesion selected as the endpoint for clinical trials against development of cervical cancer.

Although the L1 VLPs were successful, existing treatment options for HPV infection still suffer from certain drawbacks. For example, the breadth of antigenic diversity present in this group of pathogens makes induction of broadly neutralizing antibodies through current modes of treatment very difficult. L1-based compositions do not appear capable of inducing antibodies with neutralizing activities functional against a broad range of papillomavirus types. In this regard, while known L1 VLPs for treating HPV infection have high likelihood of protecting women against infection with, two, three, or even four different types of HPV, where multi-valent compositions are provided, they may not protect against infection with other types. Indeed, clinical data obtained during studies related to the efficacy of the L1 VLPs show that in control and vaccinated test groups, approximately the same number of subjects in each group developed CIN associated with HPV infection from types other than HPV-16 infection.

On the other hand, studies have indicated that the minor capsid protein (L2) appears to have some ability to induce cross-neutralizing antibodies. (See Campo (1997) Clin Dermatol; Campo (1997) Virology; Kawana (2001) Vaccine; Kawana (1998) Virology; Kawana (2003) Vaccine; Kawana (1999) J Virol; Kawana (2001) J Virol; Slupetzky (2007) Vaccine; Varsani (2003) J Virol; Gambhira (2006) Cancer Res; Gambhira (2007) J Virol; Gambhira (2007) J Virol; Kim (2008) Vaccine; Pastrana (2005) Virology; Roden (2000) Virology). Despite promising results from the perspective that cross-neutralizing antibodies could be induced, attempts at making L2-based treatment compositions were not entirely successful. They were found to be poorly immunogenic and neutralizing titers induced on administration were low. Consequently, the outcome of treatment was highly variable. (See Id).

Also among the drawbacks of existing HPV treatment options is their associated cost. For example, production of the L1 proteins in cultured cell systems, particularly those requiring use of fermentation, is expensive, e.g., using viral expression systems, such as, baculovirus expression system, a yeast expression system, or bacterial expression systems, such as an E. coli expression system. The cost of L1-VLPs currently on the market is approximate $360 for three shots, making them available to only the portion of the population in developed countries able to absorb such health-care costs, and very little, if any, of the population in developing or underdeveloped countries having few economic resources.

Accordingly, there remains a need in the art for compositions and methods for treating PV infections that address the above-identified drawbacks of existing treatment options.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compositions, comprising papillomavirus (PV) L2 polypeptides produced using a eukaryotic expression system. The presently-disclosed subject matter further includes methods of making the compositions and using the compositions for treating papillomavirus infection.

In some embodiment, a composition for treating human papillomavirus (HPV) infection in a subject susceptible to HPV infection includes an HPV L2 polypeptide produced from a eukaryotic expression system.

In some embodiments, the HPV L2 polypeptide of the composition includes a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of an HPV minor capsid (L2) protein. In some embodiments, the HPV L2 polypeptide of the composition includes a fragment extending from about amino acid 11, 12, 13, 14, or 15, and extending to about amino acid 150, 130, 120, 100, 70, or 65 of an HPV minor capsid (L2) protein.

In some embodiments, the HPV L2 polypeptide of the composition includes a fragment selected from: 5-260, 9-150, 11-130, 13-70, 13-90, 13-120, 13-150, 13-180, and 13-200. In some embodiments, the HPV L2 polypeptide comprises the 13-70 fragment. In some embodiments, the HPV L2 polypeptide comprises the 13-120 fragment. In some embodiments, the HPV L2 polypeptide comprises the 5-260 fragment. In some embodiments, the HPV L2 polypeptide comprises the 9-150 fragment. In some embodiments, the HPV L2 polypeptide comprises the 11-130 fragment.

In some embodiments, the HPV L2 polypeptide of the composition includes a fragment extending from a furin cleavage site to about amino acid 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 74, 73, 72, 71, 70, 69, 68, 67, 66, or 65 of an HPV minor capsid (L2) protein. In some embodiments, the HPV L2 polypeptide comprises a fragment extending from a furin cleavage site to a downstream amino acid between about 65 and 260 of an HPV minor capsid (L2) protein. In some embodiments, the HPV L2 polypeptide comprises a fragment extending from a furin cleavage site to a downstream amino acid between about 65 and 150. In some embodiments, the HPV L2 polypeptide comprises a fragment extending from a furin cleavage site to a downstream amino acid between about 65 and 120. In some embodiments, the HPV L2 polypeptide is from an HPV-type, selected from the group consisting of: HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82. In some embodiments, the HPV L2 polypeptide is from an HPV-type, selected from the group consisting of: HPV-6, HPV-11, HPV-16, HPV-18, HPV-31, HPV-33, HPV-35, HPV-45, HPV-52, and HPV-58. In some embodiments, the HPV L2 polypeptide is from an HPV-Type, selected from the group consisting of: HPV-6, HPV-11, HPV-16, and HPV-18.

In some embodiments, the HPV L2 polypeptide of the composition is from a first HPV-Type, and the composition is effective for treatment of infections caused by the first HPV-Type and at least one additional HPV-Type. In some embodiments, the first HPV-type is selected from an HPV-type of genus alpha-papillomavirus, and the composition is effective for treatment of the first HPV-type and at least one additional HPV-type of genus alpha-papillomavirus. In some embodiments, the first HPV-type is selected from an HPV-type of alpha-papillomavirus species 9, and the composition is effective for treatment of the first HPV-type and at least one additional HPV-type of alpha-papillomavirus species 9. In some embodiments, the first HPV-type is selected from HPV-16, 31, 33, 35, 52, 58, and 67, and the composition is effective for treatment of the first HPV-type and at least one additional HPV-type of alpha-papillomavirus species 9. In some embodiments, the first HPV-type is HPV-16, and the composition is effective for treatment of the first HPV-type and at least one additional HPV-type of alpha-papillomavirus species 9.

In some embodiments, the first HPV-type is selected from an HPV-type of alpha-papillomavirus species 7, and the composition is effective for treatment of the first HPV-type and at least one additional HPV-type of alpha-papillomavirus species 7. In some embodiments, the first HPV-type is selected from HPV-18, 45, 49, 68, 70, and the composition is effective for treatment of the first HPV-type and at least one additional HPV-type of alpha-papillomavirus species 7. In some embodiments, the first HPV-type is HPV-18, and the composition is effective for treatment of the first HPV-type and at least one additional HPV-type of alpha-papillomavirus species 7.

In some embodiments, the first HPV-Type is selected from an HPV-Type of alpha-papillomavirus species 10, and the composition is effective for treatment of the first HPV-Type and at least one additional HPV-Type of alpha-papillomavirus species 10. In some embodiments, the first HPV-Type is selected from HPV-6, 11, 13, and the composition is effective for treatment of the first HPV-Type and at least one additional HPV-Type of alpha-papillomavirus species 10. In some embodiments, the first HPV-Type is HPV-6, and the composition is effective for treatment of the first HPV-Type and at least one additional HPV-Type of alpha-papillomavirus species 10.

In some embodiments, the composition is a multi-valent composition, including at least two HPV L2 polypeptide from an HPV-type, selected from an HPV-type of alpha-papillomavirus species 9, an HPV-type of alpha-papillomavirus species 7, and an HPV-type of alpha-papillomavirus species 10, wherein each of the HPV L2 polypeptides are selected from different species. In some embodiments, the composition is a multi-valent composition, including at least three HPV L2 polypeptide from an HPV-type, selected from an HPV-type of alpha-papillomavirus species 9, an HPV-type of alpha-papillomavirus species 7, and an HPV-type of alpha-papillomavirus species 10, wherein each of the HPV L2 polypeptides are selected from different species. In some embodiments, the composition is effective for treatment of the HPV-types of alpha-papillomavirus species 9, the HPV-types of alpha-papillomavirus species 7, and the HPV-types of alpha-papillomavirus species 10. In some embodiments, a first HPV L2 polypeptide is from an HPV-type, selected from: HPV-16, 31, 33, 35, 52, 58, and 67; a second HPV L2 polypeptide is from an HPV-type, selected from: HPV-18, 45, 49, 68, 70; and a third HPV L2 polypeptide is from an HPV-type, selected from: HPV-6, 11, 13. In some embodiments, a first HPV L2 polypeptide is from HPV-16; a second HPV L2 polypeptide is from HPV-18; and a third HPV L2 polypeptide is from HPV-6.

In some embodiments, the composition comprises a fusion protein comprising the HPV L2 polypeptide. In some embodiments, the fusion protein further comprises a streptavidin (SA). In some embodiments, the HPV L2 polypeptide of the composition is conjugated to a tobacco mosaic virus (TMV).

In some embodiments, the eukaryotic expression system is a plant-based expression system. In some embodiments, the plant-based expression system comprises a tobacco mosaic virus (TMV)-based DNA plasmid.

In some embodiments, the composition includes a pharmaceutically-acceptable carrier. In some embodiments, the composition includes an adjuvant.

In some embodiments, treating the HPV infection prevents or reduces the risk of the HPV infection. In some embodiments, the composition for treating HPV infection elicits an immune response in the subject. In some embodiments, the composition for treating HPV infection prevents, reduces the risk of, ameliorates, or relieves symptoms of HPV infection. In some embodiments, the symptoms include formation of papillomas or warts, development of precancerous lesions, development of cancer, and combinations thereof.

The presently-disclosed subject matter includes a method of treating HPV infection. In some embodiments, the method of treating HPV infection includes administering an effective amount of a composition comprising an HPV L2 polypeptide produced from a eukaryotic expression system. In some embodiments, administering the effective amount of the composition immunizes against the HPV infection.

The presently-disclosed subject matter includes a composition for treating canine oral papillomavirus (COPV) infection in a subject susceptible to COPV infection. In some embodiments, the composition includes a COPV L2 polypeptide produced from a eukaryotic expression system.

In some embodiments, the COPV L2 polypeptide of the composition includes a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of COPV minor capsid (L2) protein. In some embodiments, the COPV L2 polypeptide comprises a fragment extending from about amino acid 11, 12, 13, 14, or 15, and extending to about amino acid 150, 130, 120, 100, 70, or 65 of COPV minor capsid (L2) protein. In some embodiments, the COPV L2 polypeptide comprises a fragment selected from: 5-260, 9-150, 11-130, 13-70, 13-90, 13-120, 13-150, 13-180, and 13-200. In some embodiments, the COPV L2 polypeptide comprises the 13-70 fragment. In some embodiments, the COPV L2 polypeptide comprises the 13-120 fragment. In some embodiments, the COPV L2 polypeptide comprises the 5-260 fragment. In some embodiments, the COPV L2 polypeptide comprises the 9-150 fragment. In some embodiments, the COPV L2 polypeptide comprises the 11-130 fragment.

In some embodiments, the composition comprises a fusion protein comprising the COPV L2 polypeptide. In some embodiments, the fusion protein further comprises a streptavidin (SA). In some embodiments, the COPV L2 polypeptide is conjugated to a tobacco mosaic virus (TMV).

In some embodiments, the eukaryotic expression system is a plant-based expression system. In some embodiments, the plant-based expression system comprises a tobacco mosaic virus (TMV)-based DNA plasmid.

In some embodiments, the composition includes a pharmaceutically-acceptable carrier. In some embodiments, the composition includes an adjuvant.

In some embodiments, treating the COPV infection prevents or reduces the risk of COPV infection. In some embodiments, treating the COPV infection elicits an immune response in the subject. In some embodiments, treating the COPV infection prevents, reduces the risk of, ameliorates, or relieves symptoms of HPV infection.

The presently-disclosed subject matter includes a method of treating COPV infection. In some embodiments, the method includes administering an effective amount of a composition comprising a COPV L2 polypeptide produced from a eukaryotic expression system. In some embodiments, administering the effective amount of the composition immunizes against the COPV infection.

The presently-disclosed subject matter includes a method of producing a PV L2 polypeptide in a eukaryotic expression system. In some embodiments, the method includes: identifying a PV L2 polypeptide of interest; generating an expression vector comprising a gene encoding the HPV L2 polypeptide; transcribing the gene; introducing the transcribed gene into at least one eukaryotic cell; expressing the PV L2 polypeptide from the transcribed gene within the eukaryotic cell; and isolating the PV L2 polypeptide from the eukaryotic cell.

In some embodiments, the expression vector is a tobacco mosaic virus (TMV)-based DNA plasmid. In some embodiments, the gene is under the control of a regulatory element. In some embodiments, regulatory element is a promoter. In some embodiments, the regulatory element is a T7 promoter. In some embodiments, the transcribing comprises in vitro transcription using T7 polymerase.

In some embodiments, the introducing comprises infecting the eukaryotic cell with the transcribed gene. In some embodiments, the eukaryotic cell is a Nicotiana spp. cell. In some embodiments, the eukaryotic cell is a Nicotiana benthamiana cell. In some embodiments, the eukaryotic cell is a plurality of Nicotiana spp. cells. In some embodiments, the plurality of Nicotiana spp. cells is a Nicotiana benthamiana seedling.

In some embodiments, the isolating comprises lysing the eukaryotic cell and purifying the HPV L2 polypeptide from the lysed cell.

In some embodiments, the PV L2 polypeptide is a COPV L2 polypeptide. In some embodiments, the COPV L2 polypeptide comprises a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of COPV minor capsid (L2) protein. In some embodiments, the COPV L2 polypeptide comprises a fragment extending from about amino acid 11, 12, 13, 14, or 15, and extending to about amino acid 150, 130, 120, 100, 70, or 65 of COPV minor capsid (L2) protein. In some embodiments, the COPV L2 polypeptide comprises a fragment selected from: 5-260, 9-150, 11-130, 13-70, 13-90, 13-120, 13-150, 13-180, and 13-200. In some embodiments, the COPV L2 polypeptide comprises the 13-70 fragment. In some embodiments, the COPV L2 polypeptide comprises the 13-120 fragment. In some embodiments, the COPV L2 polypeptide comprises the 5-260 fragment. In some embodiments, the COPV L2 polypeptide comprises the 9-150 fragment. In some embodiments, the COPV L2 polypeptide comprises the 11-130 fragment.

In some embodiments, the composition comprises a fusion protein comprising the COPV L2 polypeptide. In some embodiments, the fusion protein further comprises a streptavidin (SA). In some embodiments, the COPV L2 polypeptide is conjugated to a tobacco mosaic virus (TMV).

In some embodiments, the PV L2 polypeptide is a HPV L2 polypeptide. In some embodiments, the HPV L2 polypeptide comprises a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of an HPV minor capsid (L2) protein. In some embodiments, the HPV L2 polypeptide comprises a fragment extending from about amino acid 11, 12, 13, 14, or 15, and extending to about amino acid 150, 130, 120, 100, 70, or 65 of an HPV minor capsid (L2) protein. In some embodiments, the HPV L2 polypeptide comprises a fragment selected from: 5-260, 9-150, 11-130, 13-70, 13-90, 13-120, 13-150, 13-180, and 13-200. In some embodiments, the HPV L2 polypeptide comprises the 13-70 fragment. In some embodiments, the HPV L2 polypeptide comprises the 13-120 fragment. In some embodiments, the HPV L2 polypeptide comprises the 5-260 fragment. In some embodiments, the HPV L2 polypeptide comprises the 9-150 fragment. In some embodiments, the HPV L2 polypeptide comprises the 11-130 fragment.

In some embodiments, the HPV L2 polypeptide comprises a fragment extending from a furin cleavage site to about amino acid 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 74, 73, 72, 71, 70, 69, 68, 67, 66, or 65 of an HPV minor capsid (L2) protein. In some embodiments, the HPV L2 polypeptide comprises a fragment extending from a furin cleavage site to a downstream amino acid between about 65 and 260 of an HPV minor capsid (L2) protein. In some embodiments, the HPV L2 polypeptide comprises a fragment extending from a furin cleavage site to a downstream amino acid between about 65 and 150. In some embodiments, the HPV L2 polypeptide comprises a fragment extending from a furin cleavage site to a downstream amino acid between about 65 and 120.

In some embodiments, the HPV L2 polypeptide is from an HPV-type, selected from the group consisting of: HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82. In some embodiments, the HPV L2 polypeptide is from an HPV-type, selected from the group consisting of: HPV-6, HPV-11, HPV-16, HPV-18, HPV-31, HPV-33, HPV-35, HPV-45, HPV-52, and HPV-58. In some embodiments, the HPV L2 polypeptide is from an HPV-type, selected from the group consisting of: HPV-6, HPV-11, HPV-16, and HPV-18.

In some embodiments, the HPV L2 polypeptide is from a first HPV-type, and the composition is effective for treatment of infections caused by the first HPV-type and at least one additional HPV-type. In some embodiments, the first HPV-type is selected from an HPV-type of genus alpha-papillomavirus, and the composition is effective for treatment of the first HPV-type and at least one additional HPV-type of genus alpha-papillomavirus. In some embodiments, the first HPV-type is selected from an HPV-type of alpha-papillomavirus species 9, and the composition is effective for treatment of the first HPV-type and at least one additional HPV-type of alpha-papillomavirus species 9.

In some embodiments, the first HPV-type is selected from HPV-16, 31, 33, 35, 52, 58, and 67, and the composition is effective for treatment of the first HPV-type and at least one additional HPV-type of alpha-papillomavirus species 9. In some embodiments, the first HPV-type is HPV-16, and the composition is effective for treatment of the first HPV-type and at least one additional HPV-type of alpha-papillomavirus species 9.

In some embodiments, the first HPV-type is selected from an HPV-type of alpha-papillomavirus species 7, and the composition is effective for treatment of the first HPV-type and at least one additional HPV-type of alpha-papillomavirus species 7. In some embodiments, the first HPV-type is selected from HPV-18, 45, 49, 68, 70, and the composition is effective for treatment of the first HPV-type and at least one additional HPV-type of alpha-papillomavirus species 7. In some embodiments, the first HPV-type is HPV-18, and the composition is effective for treatment of the first HPV-type and at least one additional HPV-type of alpha-papillomavirus species 7.

In some embodiments, the first HPV-type is selected from an HPV-type of alpha-papillomavirus species 10, and the composition is effective for treatment of the first HPV-type and at least one additional HPV-type of alpha-papillomavirus species 10. In some embodiments, the first HPV-type is selected from HPV-6, 11, 13, and the composition is effective for treatment of the first HPV-type and at least one additional HPV-type of alpha-papillomavirus species 10. In some embodiments, the first HPV-type is HPV-6, and the composition is effective for treatment of the first HPV-type and at least one additional HPV-type of alpha-papillomavirus species 10.

In some embodiments, the composition is a multi-valent composition including at least two HPV L2 polypeptide from an HPV-type, selected from an HPV-type of alpha-papillomavirus species 9, an HPV-type of alpha-papillomavirus species 7, and an HPV-type of alpha-papillomavirus species 10, wherein each of the HPV L2 polypeptides are selected from different species. In some embodiments, the composition is a multi-valent composition including at least three HPV L2 polypeptide from an HPV-type, selected from an HPV-type of alpha-papillomavirus species 9, an HPV-type of alpha-papillomavirus species 7, and an HPV-type of alpha-papillomavirus species 10, wherein each of the HPV L2 polypeptides are selected from different species. In some embodiments, the composition is effective for treatment of the HPV-types of alpha-papillomavirus species 9, the HPV-types of alpha-papillomavirus species 7, and the HPV-types of alpha-papillomavirus species 10. In some embodiments, the first HPV L2 polypeptide is from an HPV-type, selected from: HPV-16, 31, 33, 35, 52, 58, and 67; the second HPV L2 polypeptide is from an HPV-type, selected from: HPV-18, 45, 49, 68, 70; and the third HPV L2 polypeptide is from an HPV-type, selected from: HPV-6, 11, 13. In some embodiments, the first HPV L2 polypeptide is from HPV-16; the second HPV L2 polypeptide is from HPV-18; and the third HPV L2 polypeptide is from HPV-6.

In some embodiments, the composition comprises a fusion protein comprising the HPV L2 polypeptide. In some embodiments, the fusion protein further comprises a streptavidin (SA). In some embodiments, the HPV L2 polypeptide is conjugated to a tobacco mosaic virus (TMV).

### BRIEF DESCRIPTION OF THE DRAWINGS

FIG. 1 is an amino acid sequence alignment of amino-terminal portions of COPV minor capsid (L2) protein, and HPV-6, HPV-11, HPV-16, HPV-18, HPV-26, HPV-31, HPV-35, HPV-40, HPV-45, HPV-53, and HPV58 minor capsid (L2) proteins;

FIG. 2A includes results from SDS-PAGE analysis of crude plant extract containing COPV L2₆₁₋₁₇₁:SA, where lanes 1 and 4 include Mark molecular weight marker (Invitrogen), lanes 2 and 3 include crude extract from uninfected plant tissue in Tris or 1×SDS PAGE loading dye, and lanes 5 and 6 include crude extract from plants inoculated with COPV L2₆₁₋₁₇₁:SA in Tris or 1×SDS PAGE loading dye;

FIG. 2B includes results from anti-streptavidin (SA) western blot analysis of crude plant extract containing COPV L2₆₁₋₁₇₁:SA, where lanes 1 and 2 include crude extract from uninfected plant tissue in Tris or 1×SDS PAGE loading dye, and lanes 3 and 4 include crude extract from plants expressing COPV L2₆₁₋₁₇₁:SA in Tris or 1×SDS PAGE loading dye;

FIG. 3A includes results of an ELISA study showing the reactivity of dog sera to His-tagged COPV L2, where groups of dogs were vaccinated with COPV L2₆₁₋₁₇₁ (1) or COPV L2₅₋₂₆₀ (2); serum endpoint dilutions are for the anti-COPV L2 response following three vaccinations;

FIG. 3B include results of an ELISA study showing the reactivity of dog sera to Streptavidin, where groups of dogs were vaccinated with COPV L2₆₁₋₁₇₁(1) or COPV L2₅₋₂₆₀ (2); serum endpoint dilutions are for anti-SA immune response following three vaccinations;

FIG. 4 includes results of a Western blot analysis of an initial solubility screen, using anti-streptavidin serum, where GJ (“green juice”) refers to homogenized plant tissue, S1 refers to supernatant resulting from centrifugation after pH adjustment of GJ, PEI refers to supernatant resulting from centrifugation after treating GJ with 0.4% PEI;

FIG. 5A includes SDS PAGE results of the process stream for ID 1861 (SA-COPV L2₅₋₂₆₀-His) through iminobiotin purification, where Mark 12 (Invitrogen) is used as a protein marker, and where GJ (“green juice”) refers to homogenized plant tissue, S1 refers to initial supernatant, P1 refers to initial pellet, sup & pel PEI refer to supernatant and pellet after addition of 0.1% PEI and centrifugation, sup & pel pH 11 refer to supernatant and pellet after pH adjustment of sup PEI, load refers to load onto iminobiotin column, FT refers to flowthrough, pool refers to peak fractions pooled prior to pH adjustment, and dialyzed refers to pooled fractions heated to 95° C. and 60° C. for gel analysis after dialysis using Tween 20-passivated dialysis tubing;

FIG. 5B includes SDS PAGE results of the process stream for ID 1861 (SA-COPV L2₅₋₂₆₀-His) through a polishing steps following the iminobiotin purification referenced in FIG. 5A, where load refers to dialyzed material from iminobiotin purification, FT refers to flowthrough, F## refers to eluant fractions, pel & sup refers to pellet and supernatant from ammonium sulfate pellet resuspension and re-centrifugation to remove insolubles, and 30% refers to resuspended 30% ammonium sulfate pellet;

FIG. 6 includes SDS page results for the process stream for ID 3533 (SA-HPV L2₁₁₋₁₃₀), where GJ (“green juice”) refers to homogenized plant extract, S1 refers to initial supernatant, P1 refers to initial pellet, sup & pel pH refers to supernatant and pellet after adjustment of S1 to pH 10.5 and centrifugation, L refers to load, FT refers to flowthrough, F## refers to eluant fractions, pool refers to pooled peak fractions, D refers to pooled fractions after dialysis into PBS, 95 & 60 final refer to material after 30% ammonium sulfate precipitation and re-centrifugation, and where Mark 12 (Invitrogen) is used as a protein marker;

FIG. 7A includes SDS PAGE results of a band shift analysis for the COPV L2 streptavidin fusion ID 1861 (SA-COPV L2₅₋₂₆₀-His), alone and complexed to biotinylated 1295.4 TMV capsids, where biotin was added to a 5-fold molar excess in all complexed (L2/K-TMV) samples; and

FIG. 7B includes SDS PAGE results of a band shift analysis for the HPV 16 L2 streptavidin fusion ID 3533 (SA-HPV L2₁₁₋₁₃₀), alone and complexed to biotinylated 1295.4 TMV capsids, where biotin was added to a 5-fold molar excess in all complexed (L2/K-TMV) samples.

### BRIEF DESCRIPTION OF THE SEQUENCE LISTING

SEQ ID NO: 1 is the amino acid sequence of HPV-16 minor capsid (L2) protein;

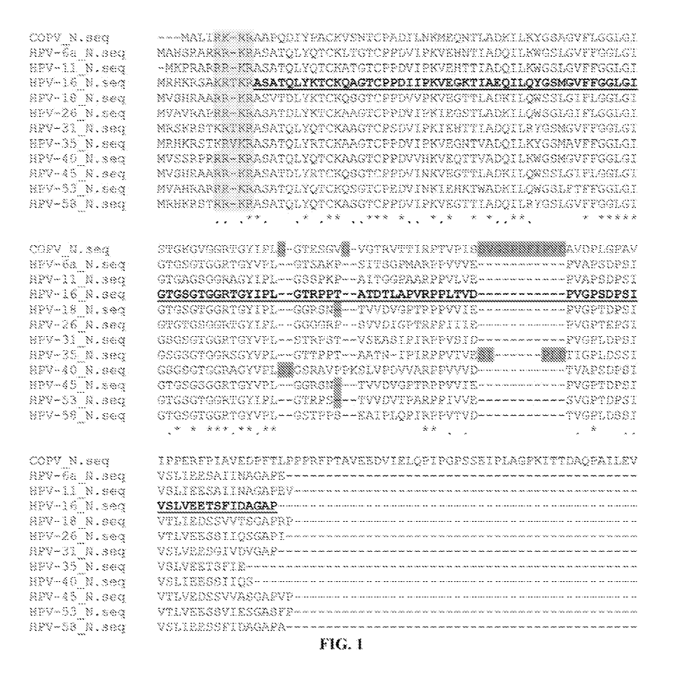

SEQ ID NO: 2 is an amino-terminal portion of the amino acid sequence of COPV minor capsid (L2) protein, as set forth in FIG. 1;

SEQ ID NO: 3 is an amino-terminal portion of the amino acid sequence of HPV-6a minor capsid (L2) protein, as set forth in FIG. 1;

SEQ ID NO: 4 is an amino-terminal portion of the amino acid sequence of HPV-11 minor capsid (L2) protein, as set forth in FIG. 1;

SEQ ID NO: 5 is an amino-terminal portion of the amino acid sequence of HPV-16 minor capsid (L2) protein, as set forth in FIG. 1;

SEQ ID NO: 6 is an amino-terminal portion of the amino acid sequence of HPV-18 minor capsid (L2) protein, as set forth in FIG. 1;

SEQ ID NO: 7 is an amino-terminal portion of the amino acid sequence of HPV-26 minor capsid (L2) protein, as set forth in FIG. 1;

SEQ ID NO: 8 is an amino-terminal portion of the amino acid sequence of HPV-31 minor capsid (L2) protein, as set forth in FIG. 1;

SEQ ID NO: 9 is an amino-terminal portion of the amino acid sequence of HPV-35 minor capsid (L2) protein, as set forth in FIG. 1;

SEQ ID NO: 10 is an amino-terminal portion of the amino acid sequence of HPV-40 minor capsid (L2) protein, as set forth in FIG. 1;

SEQ ID NO: 11 is an amino-terminal portion of the amino acid sequence of HPV-45 minor capsid (L2) protein, as set forth in FIG. 1;

SEQ ID NO: 12 is an amino-terminal portion of the amino acid sequence of HPV-53 minor capsid (L2) protein, as set forth in FIG. 1;

SEQ ID NO: 13 is an amino-terminal portion of the amino acid sequence of HPV-58 minor capsid (L2) protein, as set forth in FIG. 1;

SEQ ID NO: 14 is a cDNA sequence encoding a fusion polypeptide comprising the HPV-16 L2 amino terminal peptide sequence encompassing amino acids 13 through 92 fused at the amino terminus of the streptavidin core protein;

SEQ ID NO: 15 is a cDNA sequence encoding a fusion polypeptide comprising the HPV-16 L2 amino terminal peptide sequence encompassing amino acids 13 through 92 fused at the amino terminus of the streptavidin core protein and linked by a (Gly4Ser)₃ flexible linker sequence; and

SEQ ID NO: 16 is a HPV-16 L2 amino terminal-streptavidin fusion polypeptide encoded by SEQ ID NO: 15.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Some of the polynucleotide and polypeptide sequences disclosed herein are cross-referenced to SwissProt accession numbers in the UniProt Knowledgebase. The sequences cross-referenced to SwissProt accession numbers are expressly incorporated by reference as are equivalent and related sequences present in the UniProt Knowledgebase or other public databases. Also expressly incorporated herein by reference are all annotations present in the UniProt Knowledgebase associated with the sequences disclosed herein.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently-disclosed subject matter includes compositions, comprising papillomavirus (PV) L2 polypeptides produced using a eukaryotic expression system. The presently-disclosed subject matter further includes methods of making the compositions and using the compositions for treating papillomavirus infection.

Compositions for treating papillomavirus infection of the presently-disclosed subject matter include a papillomavirus (PV) L2 polypeptide. As used herein, a “PV L2 polypeptide” is an isolated polypeptide comprising the amino acid sequence of a full-length PV minor-capsid (L2) protein, a functional fragment thereof, or a functional variant thereof. A PV L2 polypeptide can be a canine oral papillomavirus (COPV) L2 polypeptide. A PV L2 polypeptide can be a human papillomavirus (HPV) L2 polypeptide. A COPV L2 polypeptide is an isolated polypeptide comprising the amino acid sequence of a full-length COPV minor-capsid (L2) protein, a functional fragment thereof, or a functional variant thereof. Similarly, an HPV L2 polypeptide is an isolated polypeptide comprising the amino acid sequence of a full-length HPV minor-capsid (L2) protein, a functional fragment thereof, or a functional variant thereof. An HPV L2 polypeptide can be provided from any HPV-type.

Examples of HPV-types include, but are not limited to, HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82. HPV-types can further include subtypes; for example, when HPV-6 is referenced herein, it is understood to refer to HPV-6a and HPV-6b, and other subtypes that could be discovered. The full length amino acid sequence of HPV-16 is provided as SEQ ID NO: 1. As will be understood by those of ordinary skill in the art, the amino acid and nucleotide sequences of PVs are available, for example, in the UniProt Knowledgebase, and can be accessed by searching by name or by SwissProt accession number. SwissProt accession numbers for some HPVs are as follows, and others can be easily obtained by those of ordinary skill in the art: HPV 6 L2 (Q84297); HPV 11 L2 (P04013); HPV-18 L2 (PO6793); HPV-31 L2 (P17389); HPV-33 L2 (PO6418); HPV 35 L2 (P27234); HPV-45 L2 (P36761); HPV 52 L2 (P36763); and HPV 58 L2 (P26538).

In some instances, it can be useful to describe papillomaviruses as being grouped into categories. For example, papillomaviruses can be grouped into “genera,” as set forth in de Villiers, et al., (2004) Classification of papillomavirus, Virology 324:1, pp. 17-27, which is incorporated herein by this reference. Within each genus of de Villiers, et al., are a group of so-called “species.” Each papillomavirus type can be described as being within a particular species. For example, genus alpha-papillomavirus, species 9, includes the following HPV-types: HPV-16, -31, -33, -35, -52, -58, and -67. For another example, genus alpha-papillomavirus, species 7, includes the following HPV-types: HPV-18, -45, -49, -68, and -70. For yet another example, genus alpha-papillomavirus, species 10, includes the following HPV-types: HPV-6, -11, and -13.

The term “isolated”, when used in the context of an isolated polynucleotide or an isolated polypeptide, is a polynucleotide or polypeptide that, by the hand of man, exists apart from its native environment and is therefore not a product of nature. An isolated polynucleotide or polypeptide can exist in a purified form or can exist in a non-native environment such as, for example, in a transgenic host cell.

The term “native” refers to a gene that is naturally present in the genome of an untransformed cell. Similarly, when used in the context of a polypeptide, “native” refers to a polypeptide that is encoded by a native gene of an untransformed cell's genome.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment,” when used in reference to a polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least about 50, 60, 70, 80, 90, 100, 110, 120, 130, 140, 150, 160, 170, 180, 190, 200, 225, 250, or 275 amino acids long. In some embodiments of the presently-disclosed subject matter, the fragments primarily include residues from the amino-terminal region.

A fragment can also be a “functional fragment,” in which case the fragment is capable of affecting treatment of a PV infection. In some embodiments, a functional fragment of a reference polypeptide retains some or all of the ability of the reference polypeptide to affect treatment of a PV infection. In some embodiments, a functional fragment of a reference polypeptide has an enhanced ability, relative to the reference polypeptide, to affect treatment of a PV infection. In some embodiments, the reference polypeptide is a full-length COPV L2 protein. In some embodiments, the reference polypeptide is a full-length HPV L2 protein.

The term “variant” refers to an amino acid sequence that is different from the reference polypeptide by one or more amino acids, e.g., one or more amino acid substitutions. A variant of a reference polypeptide also refers to a variant of a fragment of the reference polypeptide, for example, a fragment wherein one or more amino acid substitutions have been made relative to the reference polypeptide. A variant can also be a “functional variant,” in which case the fragment is capable of affecting treatment of a PV infection. In some embodiments, a functional variant of a reference polypeptide retains some or all of the ability of the reference polypeptide to affect treatment of a PV infection. In some embodiments, a functional variant of a reference polypeptide has an enhanced ability, relative to the reference polypeptide, to affect treatment of a PV infection. In some embodiments, the reference polypeptide is a full-length COPV L2 protein. In some embodiments, the reference polypeptide is a full-length HPV L2 protein.

The term functional variant further includes conservatively substituted variants. The term “conservatively substituted variant” refers to a polypeptide comprising an amino acid residue sequence that differs from a reference polypeptide by one or more conservative amino acid substitutions, and is capable of affecting treatment of a PV infection. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; the substitution of one acidic residue, such as aspartic acid or glutamic acid for another; or the substitution of one aromatic residue, such as phenylalanine, tyrosine, or tryptophan for another. The phrase “conservatively substituted variant” also includes polypeptides wherein a residue is replaced with a chemically derivatized residue, provided that the resulting polypeptide is capable of affecting treatment of a PV infection.

In some embodiments, the PV L2 polypeptide can be a polypeptide having 85, 86, 87, 88, 89, 90, 91, 92, 93, 94, 95, 96, 97, 98, or 99% homology to the amino acid sequence of a full-length PV minor-capsid (L2) protein, or a functional fragment thereof, so long as the resulting PV L2 polypeptide is capable of affecting treatment of a PV infection.

“Percent similarity” and “percent homology” are synonymous as herein and can be determined, for example, by comparing sequence information using the GAP computer program, available from the University of Wisconsin Geneticist Computer Group. The GAP program utilizes the alignment method of Needleman et al. (1970) J. Mol. Biol. 48:443, as revised by Smith et al. (1981) Adv. Appl. Math. 2:482. Briefly, the GAP program defines similarity as the number of aligned symbols (i.e. nucleotides or amino acids) which are similar, divided by the total number of symbols in the shorter of the two sequences. The preferred default parameters for the GAP program include: (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps. The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings

As used herein, the terms “treatment” or “treating” relate to any treatment of a PV infection, including but not limited to prophylactic treatment and therapeutic treatment As such, the terms treatment, treating, affecting treatment, and being effective for treatment include, but are not limited to: conferring protection against a PV infection; preventing a PV infection; reducing the risk of PV infection; ameliorating or relieving symptoms of a PV infection; eliciting an immune response against a PV or an antigenic component thereof; inhibiting the development or progression of a PV infection; inhibiting or preventing the onset of symptoms associated with a PV infection; reducing the severity of a PV infection; and causing a regression of a PV infection or one or more of the symptoms associated with a PV infection.

As used herein, the term “infection” refers to a colonization of a cell of a subject by a papillomavirus (PV). In some embodiments, infection refers to a colonization of a cell of the subject and an interference with normal functioning of the cell. The interference with normal functioning of the cell of the subject can result in the onset, and ultimately the expression, of symptoms in the subject.

Symptoms associated with PV infection are known to those of ordinary skill in the art and can include, but are not limited to: formation of papillomas or warts, which in infants and young children can develop into RRP; development of precancerous lesions; and development of cancer. The presence of an infection can be assessed using methods known to those or ordinary skill in the art. In some cases, the presence of a PV infection can be determined by detecting HPV DNA or RNA in a sample obtained from the subject. In some cases, the presence of a PV infection can be determined using antibodies to HPV capsid proteins, or using virus-like particles to detect serum antibodies. In some cases, non-structural proteins can be useful for detection of existing infections, and an immunoassay for the viral non-structural proteins could be useful for detection of infections in tissue samples, using techniques known to those of ordinary skillin the art, such as immunohistochemistry, western blot, or ELISA. In some cases, the presence of a PV infection can be determined by identifying a symptom associated with PV infection.

As used herein, “immunizing” and “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response can be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade and/or activation of complement) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As noted above, compositions for treating papillomavirus infection of the presently-disclosed subject matter include a PV L2 polypeptide, which can comprise or consist essentially of a functional fragment of a PV minor-capsid (L2) protein. A fragment can be identified with reference to amino acid residues in a reference polypeptide. For example, in some embodiments, a fragment can comprise or consist essentially of amino acids 13-70 of a full length HPV L2 minor capsid protein. Such a fragment can be referred to as HPV L213-70 or a 13-70 fragment.

Unless otherwise specified, a reference to a PV fragment is not specific for a particular PV or PV-type. In this regard, COPV and HPV polypeptides of different types are highly conserved, particularly in the amino-terminal portion of the polypeptides, extending from about amino acid 1 to about amino acid 260. As such, for example, a 13-70 fragment can be of interest in COPV and HPV of different types. With reference to FIG. 1, the high level of homology is illustrated in an alignment of amino-terminal portions from amino acid 1 to about amino acid 120 of COPV L2 and HPV L2 minor capsid proteins of different types. Using HPV-16 as an initial example, if a 13-120 fragment of HPV-16 (underlined in FIG. 1) is selected as a polypeptide of interest, the same region in any other alpha papillomavirus HPV L2 sequence or COPV sequence can be selected as a polypeptide of interest.

Indeed, the identified residues of 13-120 in HPV-16 would translate into the other PV types, with additions or subtractions of less than fifteen, less than ten, less than five, less than two, or no residues. For example, with continued reference to FIG. 1, the 13-120 region of HPV-16 is equivalent to the 13-120 region of HPV6, HPV-11, HPV-26, HPV-31, and HPV-58. For another example, the 13-120 region of HPV-16 is equivalent to the 13-119 region of HPV-18 because HPV-18 has one fewer amino acid (highlighted in FIG. 1) in the region. As such, when the 13-120 region of HPV-16 is of interest, the 13-119 region of HPV-18 could be of interest as well. Nevertheless, the conservation in the amino-terminal portion is such that the 13-120 region of HPV-18 could also be of interest. For another example, the 13-120 region of HPV-16 is equivalent to the 13-132 region of COPV because COPV includes 13 additional amino acids (highlighted in FIG. 1) in the region. Thus, when the 13-120 region of HPV-16 is of interest, the 13-132 region of COPV could also be of interest. Nevertheless, the conservation in the amino-terminal portion is such that the 13-120 region of COPV could also be of interest. As such, as used herein, when a PV fragment is identified, the fragment should not be considered PV-type-specific, and can be of interest in COPV L2 and any alpha papillomavirus HPV L2 sequence, unless otherwise specified.

In some embodiments, the composition comprises a PV L2 polypeptide comprising a fragment. In some embodiments, the fragment can include or consist essentially of amino acids from the amino-terminal portion of a PV minor capsid (L2) protein, for example, amino acids from the portion extending from about amino acid 1 to about amino acid 260. In some embodiments, the fragment can begin at (i.e., extend from) about amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the fragment can begin at an amino acid upstream of about amino acid 61, 60, 59, 58, 57, 56, 55, or 51. In some embodiments, the functional fragment can end at (i.e., extend to) about amino acid 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 74, 73, 72, 71, 70, 69, 68, 67, 66, or 65.

In some embodiments, the fragment begins at (i.e., extends from) about amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50; and end at (i.e., extend to) about amino acid 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 74, 73, 72, 71, 70, 69, 68, 67, 66, or 65.

In some embodiments, the composition comprises a PV L2 polypeptide comprising or consisting essentially of a PV L2 fragment selected from: 5-260, 5-250, 5-225, 5-200, 5-180, 5-175, 5-150, 5-130, 5-125, 5-120, 5-100, 5-90, 5-75, and 5-70. In some embodiments, the composition comprises a PV L2 polypeptide comprising or consisting essentially of a fragment selected from: 9-260, 9-250, 9-225, 9-200, 9-180, 9-175, 9-150, 9-130, 9-125, 9-120, 9-100, 9-90, 9-75, and 9-70. In some embodiments, the composition comprises a PV L2 polypeptide comprising or consisting essentially of a fragment selected from: 11-260, 11-250, 11-225, 11-200, 11-180, 11-175, 11-150, 11-130, 11-125, 11-120, 11-100, 11-90, 11-75, and 11-70. In some embodiments, the composition comprises a PV L2 polypeptide comprising or consisting essentially of a fragment selected from: 12-260, 12-250, 12-225, 12-200, 12-180, 12-175, 12-150, 12-130, 12-125, 12-120, 12-100, 12-90, 12-75, and 12-70. In some embodiments, the composition comprises a PV L2 polypeptide comprising or consisting essentially of a fragment selected from: 13-260, 13-250, 13-225, 13-200, 13-180, 13-175, 13-150, 13-130, 13-125, 13-120, 13-100, 13-90, 13-75, and 13-70. In some embodiments, the composition comprises a PV L2 polypeptide comprising or consisting essentially of a fragment selected from: 14-260, 14-250, 14-225, 14-200, 14-180, 14-175, 14-150, 14-130, 14-125, 14-120, 14-100, 14-90, 14-75, and 14-70. In some embodiments, the composition comprises a PV L2 polypeptide comprising or consisting essentially of a fragment selected from: 15-260, 15-250, 15-225, 15-200, 15-180, 15-175, 15-150, 15-130, 15-125, 15-120, 15-100, 15-90, 15-75, and 15-70. In some embodiments, the composition comprises a PV L2 polypeptide comprising or consisting essentially of a fragment selected from: 16-260, 16-250, 16-225, 16-200, 16-180, 16-175, 16-150, 16-130, 16-125, 16-120, 16-100, 16-90, 16-75, and 16-70.

In some embodiments, the composition comprises a PV L2 polypeptide comprising or consisting essentially of a fragment that begins at about the amino acid adjacent and downstream a furin cleavage site (See Richards, et al., (2006) PNAS, identifying the furin cleavage site of L2). The furin cleavage site of L2 is close to the amino-terminus, and follows a motif including the amino acids RXKR, where X is any amino acid. With reference to FIG. 1, the RXKR motif is highlighted for the PVs included in the alignment.

The L2 furin cleavage site for HPV-16, HPV-31, and HPV-35, for example, is between amino acids 12 and 13. As such, in some embodiments, the functional fragment is from HPV-16, HPV-31, or HPV-35 and begins at about amino acid 13. For another example, the L2 furin cleavage site for HPV-6, HPV-18, HPV-26, HPV-40, HPV-45, HPV-53, and HPV-58 is between amino acids 11 and 12. As such, in some embodiments, the functional fragment is from HPV-6, HPV-18, HPV-26, HPV-40, HPV-45, HPV-53, or HPV-58 and begins at about amino acid 12. For another example, the L2 furin cleavage site for HPV-11 is between amino acids 10 and 11. As such, in some embodiments, the fragment is from HPV-11 and begins at about amino acid 11.

As used herein with reference to a amino acid sequence, or a reference residue of an amino acid sequence, “upstream” refers to the amino acids closer to the amino-terminus of the amino acid sequence. Because the convention for presenting amino acid sequences is to present the sequence with the amino terminus to the left, writing the sequence from amino-terminus to carboxy-terminus, “upstream” refers to the amino acids to the left of a reference residue.

As used herein with reference to a amino acid sequence, or a reference residue of an amino acid sequence, “downstream” refers to the amino acids closer to the carboxy-terminus of the amino acid sequence. Because the convention for presenting amino acid sequences is to present the sequence with the carboxy-terminus to the right, writing the sequence from amino-terminus to carboxy-terminus, “downstream” refers to the amino acids to the right of a reference residue.

In some embodiments, the PV L2 polypeptide can comprise an identified fragment of a PV minor capsid (L2) protein. In this regard, the PV L2 polypeptide can include additional amino acids on one or both sides of the identified fragment, i.e., extending from the amino- and/or carboxy-terminus of the identified fragment. In some embodiments, the additional amino acids extending from the amino- and/or carboxy-terminus of the identified fragment can differ from the amino acids extending from the amino- and/or carboxy-terminus of the identified fragment in the native PV minor capsid (L2) protein. For example, in some embodiments, a PV L2 polypeptide comprising a 13-120 fragment can include additional amino acids extending from the amino-terminus of the 13-120 fragment that differ from amino acids 12 and upstream of 12 in the native PV minor capsid (L2) protein; and/or the PV L2 polypeptide comprising a 13-120 fragment can include additional amino acids extending from the carboxy-terminus of the 13-120 fragment that differ from amino acids 121 and downstream of 121 in the native PV minor capsid (L2) protein.

In some embodiments, a composition of the presently-disclosed subject matter can include a PV L2 polypeptide capable of inducing antibodies with neutralizing activities functional against a broad range of papillomavirus types. In this regard, a cross-neutralization or cross-neutralizing activity refers to the ability of a PV L2 polypeptide from a first PV-type to affect treatment of infections caused by the first PV-type and at least one additional PV-type. Without wishing to be bound by theory or mechanism, it is proposed that certain fragments of the amino-terminal portion of PV minor capsid (L2) proteins have a greater capacity for cross-neutralization, relative to the fragments of and/or including the carboxy-terminal portion of PV minor capsid (L2) proteins. This greater capacity could be due to the high level of homology among PV minor capsid (L2) proteins in the amino-terminal portion. See e.g., the alignment in FIG. 1.

In some embodiments, the composition includes a PV L2 polypeptide from a first PV-type, and the composition is effective for treatment of infections caused by the first PV-type and at least one additional PV-type. In some embodiments, the composition includes an HPV L2 polypeptide from a first HPV-type, and the composition is effective for treatment of infections caused by the first HPV-type and at least one additional HPV-type.

Without wishing to be bound by theory or mechanism, it is proposed that a first PV-type within a particular category is more likely to generate cross-neutralizing antibodies against other PV-types, if those PV-types are in the same particular category. In this regard, in some embodiments, the composition includes an HPV L2 polypeptide from a first HPV-type selected from an HPV-type of genus alpha-papillomavirus, and the composition is effective for treatment of infections caused by the first HPV-type and at least one additional HPV-type of genus alpha-papillomavirus. In some embodiments, the composition includes an HPV L2 polypeptide from a first HPV-type selected from an HPV-type of genus alpha-papillomavirus, and the composition is effective for treatment of infections caused by each of the HPV-types of genus alpha-papillomavirus.

In some embodiments, the first HPV-type is selected from an HPV-type of alpha-papillomavirus species 9, and the composition is effective for treatment of the first HPV and at least one additional HPV-Type of alpha-papillomavirus species 9. In some embodiments, the first HPV-type is selected from HPV-16, 31, 33, 35, 52, 58, and 67, and the composition is effective for treatment of the first HPV-type and at least one additional HPV-type of alpha-papillomavirus species 9 (e.g., HPV-16, 31, 33, 35, 52, 58, or 67). In some embodiments, the first HPV-type is HPV-16, and the composition is effective for treatment of HPV-16 and at least one additional HPV-type of alpha-papillomavirus species 9. In some embodiments, the first HPV-type is HPV-16, and the composition is effective for treatment of HPV-16 and HPV-31, 33, 35, 52, 58, and/or 67.

In some embodiments, the first HPV-type is selected from an HPV-type of alpha-papillomavirus species 7, and the composition is effective for treatment of the first HPV-type and at least one additional HPV-type of alpha-papillomavirus species 7. In some embodiments, the first HPV-type is selected from HPV-18, 45, 49, 68, and 70, and the composition is effective for treatment of the first HPV-type and at least one additional HPV-type of alpha-papillomavirus species 7 (e.g., HPV-18, 45, 49, 68, or 70). In some embodiments, the first HPV-type is HPV-18, and the composition is effective for treatment of HPV-18 and at least one additional HPV-type of alpha-papillomavirus species 7. In some embodiments, the first HPV-type is HPV-18, and the composition is effective for the treatment of HPV-18 and HPV-45, 49, 68, and/or 70.

In some embodiments, the first HPV-type is selected from an HPV-type of alpha-papillomavirus species 10, and the composition is effective for treatment of the first HPV-type and at least one additional HPV-type of alpha-papillomavirus species 10. In some embodiments, the first HPV-type is selected from HPV-6, 11, and 13, and the composition is effective for treatment of the first HPV-type and at least one additional HPV-type of alpha-papillomavirus species 10. In some embodiments, the first HPV-Type is HPV-6, and the composition is effective for treatment of HPV-6 and at least one additional HPV-type of alpha-papillomavirus species 10 (e.g., HPV-6, 11, or 13). In some embodiments, the first HPV-Type is HPV-6, and the composition is effective for treatment of HPV-6 and HPV-11 and/or 13.

In some embodiments, a multi-valent composition is provided for the treatment of PV infections caused by different HPV-types. A multi-valent composition refers to a composition including PV L2 polypeptides from different papillomaviruses. For example, in some embodiments, a multi-valent composition can include a first PV L2 polypeptide from HPV-16 and a second PV L2 polypeptide from HPV-18. In some embodiments, multi-valent compositions are provided for the treatment of HPV infection caused by HPVs within different categories. For example, a composition can include a first PV L2 polypeptide selected from a first category, a second PV L2 polypeptide is selected from a second category, and a third PV L2 polypeptide is selected a third category.

In some embodiments, a multi-valent composition is provided including at least two HPV L2 polypeptides from different HPV-types, selected from: an HPV-type of alpha-papillomavirus species 9; an HPV-type of alpha-papillomavirus species 7; and an HPV-type of alpha-papillomavirus species 10, where the first and second HPV L2 polypeptides are selected from different species.

In some embodiments, the multi-valent composition includes an HPV L2 polypeptide from an HPV-type of alpha-papillomavirus species 9, and an HPV L2 polypeptide from an HPV-type of alpha-papillomavirus species 7, which composition is effective for treatment of infections of each HPV-type of alpha-papillomavirus species 9 and alpha-papillomavirus species 7, including HPV-16, HPV-31, HPV-33, HPV-35, HPV-52, HPV-58, HPV-67, HPV-18, HPV-45, HPV-49, HPV-68, and HPV-70. Such a multi-valent composition could include, for example, HPV L2 polypeptides from HPV-16 and HPV-18.

In some embodiments, the multi-valent composition includes an HPV L2 polypeptide from an HPV-type of alpha-papillomavirus species 9, and an HPV L2 polypeptide from an HPV-type of alpha-papillomavirus species 10, which composition is effective for treatment of infections of each HPV-type of alpha-papillomavirus species 9 and alpha-papillomavirus species 10, including HPV-16, HPV-31, HPV-33, HPV-35, HPV-52, HPV-58, HPV-67, HPV-6, HPV-11, and HPV-13. Such a multi-valent composition could include, for example, HPV L2 polypeptides from HPV-16 and HPV-6.

In some embodiments, the multi-valent composition includes an HPV L2 polypeptide from an HPV-type of alpha-papillomavirus species 7, and an HPV L2 polypeptide from an HPV-type of alpha-papillomavirus species 10, which composition is effective for treatment of infections of each HPV-type of alpha-papillomavirus species 7 and alpha-papillomavirus species 10, including HPV-18, HPV-45, HPV-49, HPV-68, HPV-70, HPV-6, HPV-11, and HPV-13. Such a multi-valent composition could include, for example, HPV L2 polypeptides from HPV-18 and HPV-11.

In some embodiments, a multi-valent composition is provided including an HPV L2 polypeptide from an HPV-type of alpha-papillomavirus species 9, an HPV L2 polypeptide from an HPV-type of alpha-papillomavirus species 7, and an HPV L2 polypeptide from an HPV-type of alpha-papillomavirus species 10, which composition is effective for treatment of infections of each HPV-type of alpha-papillomavirus species 7, alpha-papillomavirus species 9, and alpha-papillomavirus species 10. Such a composition can be effective for treatment of infections of HPV-16, HPV-31, HPV-33, HPV-35, HPV-52, HPV-58, HPV-67, HPV-18, HPV-45, HPV-49, HPV-68, HPV-70, HPV-6, HPV-11, and HPV-13. Such a multi-valent composition could include, for example, HPV L2 polypeptides from HPV-18, HPV-16, and HPV-11.

In some embodiments of the presently-disclosed subject matter, the composition can include a PV L2 polypeptide, coupled with another molecule. In some embodiments, the composition can include a fusion protein comprising a PV L2 polypeptide. As used herein, “fusion protein” refers to a protein product of two or more genes or nucleotide sequences of interest that have been joined. Desired fusion proteins can be produced using recombinant technologies well known to those or ordinary skill in the art. In some embodiments, it can be desirable to provide a fusion protein comprising a PV L2 polypeptide and a second polypeptide of interest. In some embodiments, the composition includes a fusion protein comprising a PV L2 polypeptide and streptavidin (SA), or a desired fragment thereof. In some embodiments, a fusion protein can comprise a PV L2 polypeptide and histidine tag. In some embodiments, the composition can include a fusion protein comprising a PV L2 polypeptide, a histidine tag, and streptavidin (SA), or a desired fragment thereof. In some embodiments of the presently-disclosed subject matter, the composition can include a PV L2 polypeptide conjugated to a tobacco mosaic virus (TMV).

Compositions of the presently-disclosed subject matter can further include a pharmaceutically-acceptable carrier. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. The pharmaceutically acceptable carriers or vehicles or excipients are well known to those of ordinary skill in the art. For example, a pharmaceutically acceptable carrier or vehicle or excipient can be a NaCl (e.g., saline) solution or a phosphate buffer. The pharmaceutically acceptable carrier or vehicle or excipients can be any compound or combination of compounds facilitating the administration of the composition; advantageously, the carrier, vehicle or excipient can facilitate administration, delivery and/or improve preservation of the composition.

Compositions of the presently-disclosed subject matter can include one or more adjuvants. Suitable adjuvants include, but are not limited to: polymers of acrylic or methacrylic acid, maleic anhydride and alkenyl derivative polymers; immunostimulating sequences (ISS), such as oligodeoxyribonucleotide sequences having one or more non-methylated CpG units (See Klinman et al., Proc. Natl. Acad. Sci., USA, 1996, 93, 2879-2883; WO98/16247); an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same reference; cation lipids containing a quaternary ammonium salt; cytokines, aluminum hydroxide, aluminum phosphate, aluminum sulfate, or other alum adjuvant; other adjuvants discussed in any document cited and incorporated by reference into the instant application; or any combinations or mixtures thereof.

The oil in water emulsion, which can be particularly appropriate for viral vaccines, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil can be used in combination with emulsifiers to form an emulsion. The emulsifiers can be nonionic surfactants, such as: esters of, on the one hand, sorbitan, mannide (e.g. anhydromannitol oleate), glycerol, polyglycerol or propylene glycol and, on the other hand, oleic, isostearic, ricinoleic or hydroxystearic acids, the esters being optionally ethoxylated, or polyoxypropylene-polyoxyethylene copolymer blocks, such as Pluronic, e.g., L121.

Among the type (1) adjuvant polymers, preference is given to polymers of crosslinked acrylic or methacrylic acid, especially crosslinked by polyalkenyl ethers of sugars or polyalcohols. One or ordinary skill in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name CARBOPOL™ (BF Goodrich, Ohio, USA) are also suitable. They are crosslinked by allyl saccharose or by allyl pentaerythritol.

As to the maleic anhydride-alkenyl derivative copolymers, preference is given to EMA (Monsanto), which are straight-chain or cross-linked ethylene-maleic anhydride copolymers and they are, for example, cross-linked by divinyl ether. Reference is also made to J. Fields et al., Nature 186: 778-780, Jun. 4, 1960.

The presently-disclosed subject matter includes methods of treating PV infection in a subject. In some embodiments, the method includes administering an effective amount of a composition comprising a PV L2 polypeptide, as described above. As used herein, the term “effective amount” refers to a dosage or a series of dosages sufficient to affect treatment for a PV infection in a subject. This can vary depending on the subject, the PV (e.g., PV-type) and the particular treatment being affected. The exact amount that is required will vary from subject to subject, depending on the species, age, and general condition of the subject, the particular adjuvant being used, administration protocol, and the like. As such, the effective amount will vary based on the particular circumstances, and an appropriate effective amount can be determined in a particular case by one of ordinary skill in the art using only routine experimentation.

Administration protocols can be optimized using procedures generally known in the art. A single dose can be administered to a subject, or alternatively, two or more inoculations can take place with intervals of several weeks to several months. The extent and nature of the immune responses induced in the subject can be assessed using a variety of techniques generally known in the art. For example, sera can be collected from the subject and tested, for example, for PV DNA or RNA in a sera sample, detecting the presence of antibodies to PV or antigenic fragments thereof using, for example, PV VLPs, or monitoring a symptom associated with PV infection. Relevant techniques are well described in the art, e.g., Coligan et al. Current Protocols in Immunology, John Wiley & Sons Inc. (1994), which is incorporated herein by this reference.

As used herein, the term subject refers to both human and animal subjects. Thus, veterinary therapeutic uses are provided in accordance with the presently-disclosed subject matter. A subject susceptible to an HPV infection can be a human subject. A subject susceptible to a COPV infection can be a canine subject.

The presently-disclosed subject matter includes a method of producing an HPV L2 polypeptide in a eukaryotic expression system. Eukaryotic expression systems include plant-based systems; insect cell systems via recombinant baculoviruses; whole insect systems via recombinant baculoviruses; genetically engineered yeast systems, including but not limited to Saccharomyces sp. and Picchia spp.; and mammalian cell systems, including but not limited to Chinese hamster ovary cells or other cell lines commonly used for industrial scale expression of recombinant proteins. In some embodiments, useful plant-based expression systems can include transgenic plant systems. In some embodiments, useful plant-based expression systems can include transplastomic plant systems. In some embodiments, the GENEWARE® plant-based expression can be used.

It has been determined that a variety of different plant viruses can accept the insertion of foreign genes into their genome, without jeopardizing either the antigenic nature of the product of the inserted gene or the major functions of the recombinant virus. This has led to the development of plant virus-derived vectors for the transient expression of foreign genes in plants. A number of different viruses including Alfalfa mosaic virus (AIMV); Cowpea mosaic virus (CPMV); Plum pox virus (PPV); Potato virus X (PVX); Tomato bushy stunt virus (TBSV); and Tobacco mosaic virus (TMV) have been genetically engineered to retain their capability of infecting their natural plant hosts in addition to replicating and expressing the inserted gene of interest. The development of plant virus vectors has become a useful tool for the transient expression of foreign proteins in plants and has certain advantages over the use of transgenic plant production systems.

The use of recombinant plant viruses allows for systemic expression of a protein throughout an entire plant. An advantage displayed by the use of virus vectors is the rapidly obtainable yield of the recombinant protein. Protein expression levels are not subject to unfavorable gene insertion into the plant genome such as gene silencing or instability of the plant genes. Plant virus-derived vector systems have enhanced methods of producing proteins of interest, by enhancing the yield of desired proteins, as well as the time and cost associated with producing the desired proteins. Expression of foreign proteins in tobacco plants, for example, has been found to be particularly useful. Techniques and systems for expressing foreign proteins in tobacco plants are described in the following references, each of which are incorporated herein by reference: United States Patent Application Publication Nos. 2005/0175590 of Fitzmaurice, et al.; 2005/0009012 of Holzberg, et al.; 2004/0088757 of Roberts, et al.; 2004/0064855 of Pogue, et al.; 2003/0108557 of Garger, et al.; and 2003/0097683 of Lindbo, et al.; and U.S. Pat. Nos. 6,800,748 to Holzberg et al.; 6,720,183 to Kumagai et al.; 6,700,040 to Roberts et al.; 6,660,500 to Turpen et al.; 6,656,726 to Fitzmaurice et al.; 6,479,291 to Kumagai et al.; 6,462,255 to Turpen; 6,376,752 to Kumagai et al.; 6,300,134 to Lindbo et al.; 6,300,133 to Lindbo et al.; 5,977,438 to Turpen et al.; 5,965,794 to Turpen; 5,922,602 to Kumagai et al.; 5,889,191 to Turpen; 5,889,190 to Donson et al.; 5,866,785 to Donson et al.; 5,811,653 to Turpen; 5,589,367 to Donson et al.; and 5,316,931 to Donson et al.

Such tobacco plant systems can make use of Tobacco mosaic virus (TMV). TMV is a member of the alpha-like super family, was the first plant virus to be purified, and was the first plant virus to have its structure, genome sequence, and gene function resolved. TMV belongs to the class of positive sense single-stranded RNA viruses. The viral genome is approximately 6395 nucleotides in length and encodes a total of four open reading frames, three of which encode nonstructural proteins. The viral RNA is encapsulated in a helically arranged coat of 2160 copies of a structural protein, the coat protein (CP). In some embodiments, PV L2 polypeptides can be produced using a tobacco plant system, in accordance with methods of the presently-disclosed subject matter.

In some embodiments, a method of producing an HPV L2 polypeptide in a eukaryotic expression system includes: identifying an HPV L2 polypeptide of interest; generating an expression vector comprising a gene encoding the HPV L2 polypeptide; transcribing the gene; introducing the transcribed gene into at least one eukaryotic cell; expressing the HPV L2 polypeptide from the transcribed gene within the eukaryotic cell; and isolating the HPV L2 polypeptide from the eukaryotic cell.

With regard to the expression vector, in some embodiments, it is a tobacco mosaic virus (TMV)-based DNA plasmid.

With regard to the step of transcribing the gene, in some embodiments, the gene is under the control of a regulatory element. In some embodiments, the regulatory element can be a promoter, e.g., a T7 promoter. In some embodiments, the transcription includes in vitro transcription using T7 polymerase.

With regard to the step of introducing the transcribed gene into a eukaryotic cell, in some embodiments, the transcribed gene is introduced by infecting the eukaryotic cell with the transcribed gene, which can be an infectious RNA polynucleotide. In some embodiments, the eukaryotic cell is a Nicotiana benthamiana cell. In some embodiments, the eukaryotic cell is a plurality of Nicotiana benthamiana cells. In some embodiments, the plurality of Nicotiana benthamiana cells is a Nicotiana benthamiana seedling.

With regard to the step of isolating the PV-L2 polypeptide, in some embodiments, the isolation includes lysing the eukaryotic cell and purifying the PV L2 polypeptide from the lysed cell. Lysis is performed under neutral to alkaline conditions to facilitate obtaining the PV-L2 polypeptide in a soluble form and to minize the extent of proteolytic degradation. To aid in the precipitation and removal of host proteins and pigments, the lyzed cells or a supernatant obtained following centrifugation is treated with the cationic polymer polyethyleneimine (PEI). Optimal PEI concentration is PV-L2 polypeptide-dependent with the highest possible concentration under which the polypeptide retains solubility being chosen. This typically corresponds to a concentration in the range of about 0.05% to about 0.4% w/v. Isolation of the PV-L2 polypeptide from the PEI-treated supernatant is performed by chromatography, with affinity separation being a preferred embodiment. Ammonium sulfate prepcipitation can be incorporated into the protocol, prior to or following the chromatography, to concentrate and/or further purify the PV-L2 polypeptide through selective precipitation. For example, the ammonium sulfate concentration can be tuned to precipitate the full length PV-L2 polypeptide while truncated derivitaives remain soluble, or via versa. Dialysis on the isolated PV-L2 polypeptide places to product in its final buffer formulation.

Additional information related to methods of producing an HPV L2 polypeptide in a eukaryotic expression system can be found in the Examples set forth herein.

### EXAMPLES

The presently-disclosed subject matter is further illustrated by the following specific but non-limiting examples. The following examples may include compilations of data that are representative of data gathered at various times during the course of development and experimentation related to the present invention. Further, one or more of the following examples may be prophetic, notwithstanding numerical values, results and/or data referred to and contained in the examples.

### Example 1

### Canine Oral Papillomavirus (COPV) Model

Canine papillomas were one of the first animal systems studied to develop vaccines against PVs. Dogs are now commonly used as animal models for a variety of humans diseases. Papillomas affect many anatomic locations in dogs, similar to the human diseases. Puppies may have marginal papillae on their tongues which are normal anatomic structures resembling oral papillomas. True papillomas can be found on the dorsal tongue and buccal mucosa, ocular mucous membranes, mucous membranes of the lower genital tracts of both males and females, and haired skin. The lesions are characterized by epithelial proliferation on thin fibrovascular stalks and there may be specific cytopathic effects in the stratum granulosum in which the cells swell, develop large keratohyalin-like granules, and may have intranuclear inclusions. Group-specific papillomavirus antigens can be detected by the cells exhibiting cytopathic effects by immunohistochemistry.

The COPV model is a useful preclinical model for HPVs, for a number of reasons. Because of the high level of similarity between COPV and HPV at the polynucleotide and polypeptide sequence levels, genetic organizational level, as well as similar mucosal route of infection, COPV provides a highly suitable in vivo model for study of HPV vaccines. For example, dogs can be inoculated with compositions including COPV L2 polypeptides and challenged with live COPV in order to provide relevant in vivo evidence regarding the effectiveness of PV L2 polypeptides (e.g., PV L2 polypeptides produced from a eukaryotic expression system as disclosed herein) to confer protection against the corresponding papillomavirus.

The COPV is also important in its own right. COPV is a mucosal papillomavirus which results in papillomas in canines that are found in the dorsal tongue and buccal mucosa, ocular mucous membranes, mucous membranes of the lower genital tracts of both males and females, and haired skin. Moreover, COPV is believed to play a role in squamous cell carcinoma. Therefore, a composition effective against COPV is highly desirable because it may be used to prevent papillomas in canines, and also squamous carcinoma caused by COPV.

Additionally, given the substantial similarities between COPV and HPVs, the COPV/beagle animal model has applicability in screening the effectiveness of potential antiviral compositions for treating human papillomavirus infection. For example, briefly, this can involve administering an antiviral composition predicted to be useful for treating human papillomavirus infection to a beagle dog which has been infected with COPV and determining the effects of this antiviral agent on the status of COPV infection. Effects can be determined, for example, by observing the size and number of papillomas in the treated animal before and after treatment with the antiviral composition. Antiviral compositions that inhibit papilloma development or result in their decrease in size and/or number in treated animals should possess similar activity in humans for treating HPV infection, given the similarities between COPV and HPVs.

Furthermore, the COPV model is the established preclinical model for efficacy studies for cervical cancer and papillomavirus infection treatment compositions. The COPV animal model was used in the preclinical studies related to the L1-based human cervical cancer compositions now on the market. Using the COPV model, the L1-based composition was found to be about 100% effective. Clinical trials were thereafter conducted, producing substantially the same results as the COPV studies, confirming the utility of the COPV model in this context. Information related to L1-based compositions, for example, the compositions known as GARDASIL® (Merck & Co., Inc. Whitehouse Station, N.J., USA), and CERVARIX® (GlaxoSmithKline, Middlesex, United Kingdom), can be found in the following references, each of which are incorporated herein by reference: U.S. Pat. Nos. 7,001,995 to Neeper et al.; 6,908,615 to Hofmann et al.; 6,887,478 to Schlegel, et al.; 6,165,471 to Garcea et al.; 6,153,201 to Rose et al.; 5,643,765 to Willey; 5,639,606 to Willey; and 5,283,171 to Manos et al.; and United States Patent Application Publication No. 2005/0026257 of Gissmann, et al.

The COPV model was used for studies described herein. Endogenous COPV causes oral papillomas in up to about 10% of weanling beagles and is necessary to induce malignant transformation in the about 5% of papillomas that do not spontaneously regress. COPV studies were conducted in weanling beagles using different PV L2 polypeptides.

### Example 2

### Plant-Produced PV L2 Polypeptides, PV L2₆₁₋₁₇₁ and PV L2₅₋₂₆₀

PV L2 polypeptides were produced in Nicotiana benthamiana using a tobacco mosaic virus (TMV)-based gene expression system (Smith, et al. 2006. Virology), purified, and administered to beagle dogs. Animals received compositions including the PV L2 polypeptides three times, at two week intervals. Study endpoints included serology; analysis of COPV neutralizing titers in a pseudovirus-based neutralization assay using reagents developed by the inventors; and development of oral papillomas after challenge with a high titer stock of infectious COPV. All treated animals produced antibodies to L2 and the streptavidin (SA) carrier protein. The COPV L2₅₋₂₆₀ composition induced good levels of neutralizing antibodies and protected all 4 vaccinated animals against challenge with COPV, while the COPV L2₆₁₋₁₇₁ vaccine induced protective immunity in 2 out of 4 vaccinated animals. The degree of protection against challenge in the COPVL2₆₁₋₁₇₁ cohort was correlated with L2-reactive antibody titers. Two L1 composition-vaccinated animals were protected from challenge, as expected, and the two mock-vaccinated animals developed large oral warts. The data described herein indicate that a composition including a PV L2 polypeptide produced in a eukaryotic expression system has utility for treating, including preventing, papillomavirus infection.

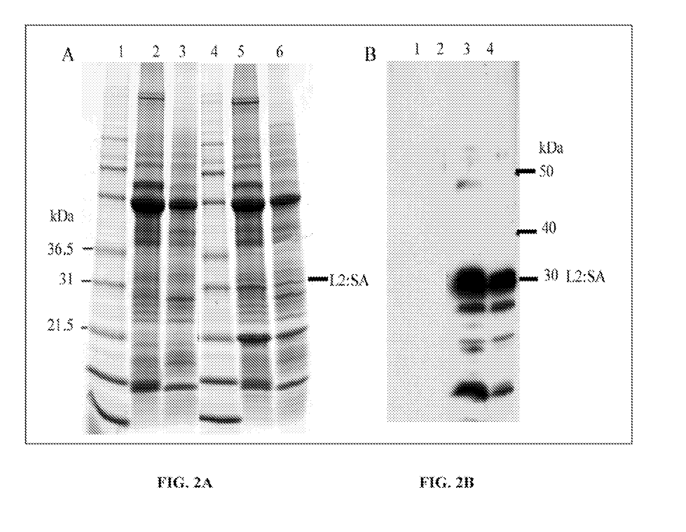

Manufacture and characterization of soluble PV L2 proteins. N. benthamiana plants expressing COPV L2₆₁₋₁₇₁ by using a modified tobacco mosaic virus (TMV) expression vector displayed the expected mosaic phenotype, and systemically infected tissue was harvested 7 days post infection. With reference to FIG. 2A, the expression of COPV L2₆₁₋₁₇₁ was detected in crude plant extracts, migrating as a band with an apparent molecular weight of 30 kDa by SDS-PAGE. With reference to FIG. 2B, to confirm the identity of the putative COPV L2₆₁₋₁₇₁:SA band, a western blot was performed on the same set of crude extracts. COPV L2₆₁₋₁₇₁:SA extracts showed strong reactive bands near the 30 kDa marker, and no signal was present in the negative control samples. The 30 kDa band is close to the predicted molecular weight of the fusion (25 kDa). The observed size difference may reflect some post-translational modifications occurring in planta or the conformation of the fusion may retard electrophoretic mobility. In addition to the 30 kDa band, there are other smaller minor bands that may represent multiple truncations of the full COPV L2₆₁₋₁₇₁:SA fusion. The overall yield of purified COPV L2₆₁₋₁₇₁ corresponds to approximately 60-120 mg/kg plant tissue.

N. benthamiana plants expressing COPV L2₅₋₂₆₀ also displayed the expected mosaic phenotype, and systemically infected tissue was harvested 6 or 7 days post infection. Expression of COPV L2₅₋₂₆₀ was similarly evaluated under acidic (50 mM sodium acetate, pH 5) and alkaline (50 mM Tris, pH 8) conditions by SDS PAGE and western blot. A faint band corresponding to COPV L2₅₋₂₆₀ was visible for the crude extracts by SDS PAGE, with an apparent molecular weight of 45-50 kDa. A reactive band was observed by western blot. The final yield of purified COPV L2₅₋₂₆₀ was 130-150 mg/kg plant tissue.

Production of soluble PV L2-streptavidin fusion proteins. Two L2 polypeptides were selected as examples for use in the present example. Two fragments of the COPV L2 protein, COPV L2₆₁₋₁₇₁ and COPV L2₅₋₂₆₀, were fused to the SA protein, which acted as a carrier and affinity tag for the treatment composition. Briefly, these constructs were expressed in planta using a modified tobacco mosaic virus (TMV) viral vector. (See Smith, et al. (2006) Virology), and the proteins were extracted as described below.

Purified Streptomyces avidinii genomic DNA was purchased from the American Tissue Culture Collection (ATCC; Manassas, Va., U.S.A.) and the coding sequence for SA core, corresponding to amino acids 40-163 of Swissprot accession number P22629, was amplified by PCR. The resulting fragment was ligated into a Sad site on the 3′ end of a GFP insert in p30B GFPc3, a TMV expression vector (Shivprasad et al., 1999). This vector was subsequently modified to remove the GFP and introduce NgoMIV and AvrII restriction sites at the N-terminus of SA core to generate the plasmid pLSB1821. COPV L2 (Genbank accession number NP056818) was synthesized by Geneart (Regensburg, Germany) using the preferred codon usage for tobacco. DNA fragments corresponding to either amino acids 61-171 or 5-260 were digested with NgoMIV and AvrII and cloned into pLSB1821, to generate COPV L2₆₁₋₁₇₁ and COPV L2₅₋₂₆₀, respectively. Infectious transcripts were generated using the MMESSAGE MMACHINE® kit (Ambion, Austin, Tex., U.S.A.) and inoculated onto N. benthamiana plants.

To isolate COPV L2₆₁₋₁₇₁, plant tissue was homogenized in 3 volumes of extraction buffer (25 mM Tris-maleic acid buffer, pH 7.5, 0.01% w/v sodium metabisulfite). Following addition of 0.4% (w/v) polyethyleneimine (PEI), the homogenate was centrifuged and a 30% ammonium sulfate cut was performed on the clarified supernatant, and the resulting pellet was resuspended. Affinity chromatography was performed using an AKTA purifier (Amersham Biosciences, Piscataway, N.J., U.S.A.). The clarified extract was adjusted to pH 11 and loaded onto an immobilized imminobiotin column (Pierce, Rockford, Ill., U.S.A.). The fusion was eluted with 0.1 M acetic acid, pH 4.0, and the peak fractions were concentrated with a 50% ammonium sulfate cut, followed by resuspension in phosphate-buffered saline, pH 7.4 (Invitrogen, Carlsbad, Calif., U.S.A.).

To extract COPV L2₅₋₂₆₀, infected tissue was homogenized in 3 volumes of extraction buffer (50 mM Tris, 10 mM EDTA, 0.04% w/v sodium metabisulfite). The homogenate was adjusted to pH 7.2-7.8, followed by centrifugation at 10,000×g for 10 minutes. 0.1% v/v PEI was added to the supernatant and centrifugation was repeated. The resulting supernatant was adjusted to pH 10.5 and the non-proteinaceous precipitate that formed was removed by centrifugation at 10,000×g for 10 minutes. The clarified supernatant was sequentially filtered through 1.0 um glass fiber and 0.45 um filters prior to iminobiotin chromatography. After chromatography, the peak fractions were combined, adjusted to pH 9, and dialyzed into 1×PBS using Tween-20 passivated dialysis tubing. The dialyzed fusion was then adjusted to 10 mM imidazole in preparation for polishing with IMAC chromatography (Amersham). The peak fractions were combined and concentrated by a 30% ammonium sulfate cut. The pellet was resuspended in 1×PBS.

Polyacrylamide gel electrophoresis (PAGE) of proteins in the presence of sodium dodecyl sulfate (SDS) (Laemmli, 1970) was performed on 10-20% Tris-glycine gels (BioRad, Hercules, Calif., U.S.A. or Invitrogen) according to the manufacturer's instructions. The MARK 12™ or MAGIC MARK XP™ protein standards (both Invitrogen) were employed as molecular weight references. Western blots (Towbin et al., 1979), employing 0.2 um polyvinylidene fluoride membrane (PVDF) (BioRad) or nitrocellulose (x), were probed with rabbit anti-streptavidin polyclonal (Sigma, St. Louis, Mo., U.S.A.). The secondary antibody was a goat anti-rabbit horseradish peroxidase (HRP) (Bio-Rad) or goat anti-rabbit alkaline-phosphatase (AP) (Sigma). HRP detection was with the ECL+chemiluminescent kit (Amersham) with Hyperfilm ECL (Amersham) employed for image capture, as per the manufacturer's instructions. AP detection was with the BCIP/NBT kit (Invitrogen).

Vaccine Groups. Groups of beagle weanlings, containing one to four dogs per group, were used for treatment and challenge experiments. The beagles received compositions formulated in RIBI adjuvant (Corixa Corp., Hamilton, Mont., U.S.A.). As set forth in Table 1, a first group received COPV L2₆₁₋₁₇₁, a second group received COPV L2₅₋₂₆₀, a third group received phosphate-buffered saline (PBS) as a negative control, a fourth group received TMV alone, as a negative control, and a fifth group received an L1 composition, as described in Suzich, et al. 1995, as a positive control. The study was approved by the Institutional Animal Care and Use Committee for the University of Louisville.

TABLE 1
Vaccine groups

| Group | Number of dogs | Composition |
|---|---|---|
| Test Dogs | 4 | COPV L2₆₁₋₁₇₁/SA |
| Test Dogs | 4 | COPV L2₅₋₂₆₀/SA/His |
| Negative Control Dogs | 1 | PBS |
| Negative Control Dogs | 2 | TMV |
| Positive Control Dog | 2 | L1 |

Immunizations and Viral Challenge. Vaccines were prepared in RIBI adjuvant at doses of 500 ug of PV L2 polypeptide per milliliter of phosphate-buffered saline. Groups of dogs were immunized with 150 ug PV L2 polypeptide, except one dog which received 75 ug of COPV L2₆₁₋₁₇₁, by subcutaneous injection in the dew claw. Dogs receiving positive control treatment were administered 5 μg of the L1 composition of Suzich et al. 1995. Dogs received three administrations at two-week intervals (Days 0, 17, 31). Two weeks after the final administration, dogs were infected with COPV as described in Suzich et al., 1995. Beagles were infected by gentle abrasion of the buccal mucosa bilaterally, followed by application of an undiluted COPV wart homogenate, at two sites per dog. The multiple treatment site used on each dog allows for a statistically-significant determination of efficacy. That is to say that, in some embodiment, all sites on all dogs within a test group must be negative for development of papillomas in order for efficacy to be found. Dogs were monitored weekly until the first warts appeared, then several times per week following wart development.

Serum antibody analyses (ELISA). Pre-administration and post-administration sera were collected and evaluated for neutralization. Beagle sera were prepared by centrifugation from blood obtained from the jugular or cephalic veins on Days 0 (pre-bleed), 17, 31, and 45. Multi (96)-well microtiter plates (Maxisorp, Nalge Nunc International, Rochester, N.Y., U.S.A.) were coated with bacterially-expressed His-tagged COPV L2 or streptavidin (Sigma), in carbonate/bicarbonate buffer (pH 9.6). After blocking with casein, serial dilutions of the sera were added. Following incubation, the plates were washed and incubated with goat anti-dog HRP-conjugated secondary antibody (Bethyl Laboratories, Montgomery, Tex., U.S.A.). Plates were developed using tetramethyl benzidine substrate solution (KPL, Gaithersburg, Md., U.S.A.), and the reactions stopped by addition of 0.6 N sulfuric acid. Plate absorbance was read at 450 nM in a 96-well plate spectrophotometer (Spectra MR, Dynex Technologies, Chantilly, Va., U.S.A.). The serum endpoint dilution corresponded to an absorbance reading of twice background. Data analysis was performed using GraphPad Prism, version 4.03 (San Diego, Calif., U.S.A.).

Virus neutralization assay. Pre-administration and post-administration sera were collected and evaluated for neutralization. Sera from dogs were tested for neutralization of COPV using a pseudovirus neutralization assay as described by Pastrana et al., 2004, with some minor modifications. Microcultures of 293FT cells (Invitrogen) were plated into wells of a 96-well microtiter plate. Two to five hours later, aliquots of COPV pseudovirion, incubated on ice for one hour with dilutions of dog sera, were added to the wells in triplicate and the plates cultured for three days at 37° C. Cell culture supernatants were assessed for alkaline phosphatase activity by using the Great ESCAPE™ SEAP kit following the manufacturer's instructions (Clontech, Mountain View, Calif., U.S.A.). Neutralization titers were defined as the dilution of sera that reduced OD values to 50% of maximum values derived from cultures receiving pseudovirions alone. All neutralization assays were performed multiple times.

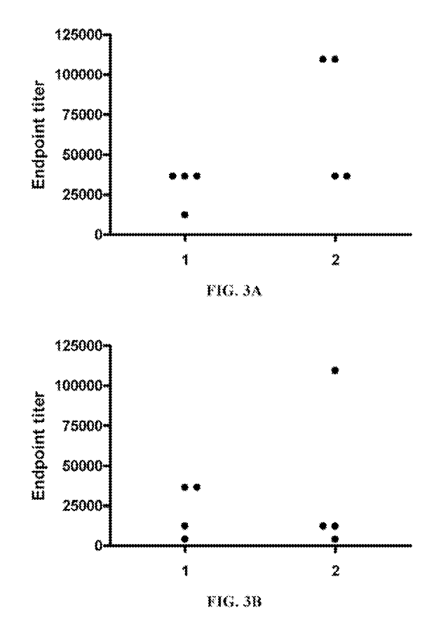

ELISA Results. Post-vaccination sera was collected and assessed for antibodies to histadine-tagged COPV L2 and to streptavidin. Pre-immune sera were pooled and used as a negative control. The data were plotted as the individual endpoint titers with values greater than twice background. With reference to FIG. 3A, ELISA reactivity to COPV L2 was higher on average for the animals vaccinated with COPV L₂₅₋₂₆₀. With reference to FIG. 3B, reactivity to streptavidin was similar for both the COPV L2₆₁₋₁₇₁ and COPV L2₅₋₂₆₀ vaccinated animals, with one high-responder in the COPV L2₅₋₂₆₀ group.

Neutralization of COPV pseudovirus. COPV pseudovirions were tested to determine whether the L2 vaccines induced neutralizing antibodies. Dogs vaccinated with COPV L2₆₁₋₁₇₁ did not strongly neutralize the pseudovirus, with half-maximal titers of 40 or less. Dogs vaccinated with COPV L2₅₋₂₆₀ showed strong neutralizing titers that ranged from 160 to 640. Results are described in Table 2.

TABLE 2
Neutralization of COPV Pseudovirus

| Animal | Composition | Endpoint Dilution Titers |
|---|---|---|
| Negative Control Dogs | Saline or TMV | 0 |
| Positive Control Dog | L1 Composition | 0 |
| Test Dog 1 | COPV L2₆₁₋₁₇₁/SA | 20 |
| Test Dog 2 | COPV L2₆₁₋₁₇₁/SA | 40 |
| Test Dog 3 | COPV L2₆₁₋₁₇₁/SA | <40 |
| Test Dog 4 | COPV L2₆₁₋₁₇₁/SA | <40 |
| Test Dog 5 | COPV L2₅₋₂₆₀/SA/His | 640 |
| Test Dog 6 | COPV L2₅₋₂₆₀/SA/His | 640 |
| Test Dog 7 | COPV L2₅₋₂₆₀/SA/His | 160 |
| Test Dog 8 | COPV L2₅₋₂₆₀/SA/His | 640 |

Outcome of administration with PV L2 Polypeptides in Dogs Challenged with COPV. Dogs receiving the PV L2 polypeptides were challenged with COPV wart homogenate as described above. Animals that received the COPV L2₆₁₋₁₇₁ composition developed warts at 3 of 8 sites, with one animal developing warts at both challenge sites and one dog developing warts at one site. Dogs receiving the COPV L2₅₋₂₆₀ vaccine were completely protected from viral challenge. Negative control dogs, vaccinated with either PBS or TMV, developed warts at both challenge sites; positive control dogs, vaccinated with the L1 composition, were completely protected. The results are set forth in Table 3.

TABLE 3

| | | Warts |
|---|---|---|
| Animal | Composition/Saline | (left site/right site) |
| Negative Control Dog | Saline | Warts on both sites (+/+) |
| Negative Control Dog | TMV | Warts on both sites (+/+) |
| Positive Control Dog | L1 Composition | No warts (−/−) |
| Test Dog 1 | COPV L2₆₁₋₁₇₁/SA | No warts (−/−) |
| Test Dog 2 | COPV L2₆₁₋₁₇₁/SA | No warts (−/−) |
| Test Dog 3 | COPV L2₆₁₋₁₇₁/SA | Warts on one site (+/−) |
| Test Dog 4 | COPV L2₆₁₋₁₇₁/SA | Warts on both sites (+/+) |
| Test Dog 5 | COPV L2₅₋₂₆₀/SA/His | No warts (−/−) |
| Test Dog 6 | COPV L2₅₋₂₆₀/SA/His | No warts (−/−) |
| Test Dog 7 | COPV L2₅₋₂₆₀/SA/His | No warts (−/−) |
| Test Dog 8 | COPV L2₅₋₂₆₀/SA/His | No warts (−/−) |

The data disclosed in the present example indicate that a composition including a PV L2 polypeptide producing in a eukaryotic expression system has utility for treating papillomavirus infection.

The minor capsid protein (L2) of human papillomavirus (HPV) contains epitopes that can induce antibodies with cross-neutralizing activity, indicating that a PV L2 polypeptide composition can potentially protect against the multiple, e.g., 13 or more oncogenic HPV types implicated in the etiology of cervical cancer. Furthermore, expression of PV L2 polypeptides in plant systems offers the potential for production of appropriately-folded viral antigens, at low cost and at agricultural scale.

### Example 3

### Plant-Produced PV L2 Polypeptide, COPV L2₆₁₋₁₇₁

The present example included a positive control dog inoculated with an L1 composition as described in Suzich, et al. 1995, and a negative control animal inoculated saline or tobacco mosaic virus (TMV), alone. In the present example, the positive control animals were wart free, and the negative control animals developed large, confluent oral warts on both of the buccal mucosa.

Tobacco plant-produced PV L2 polypeptide including amino acid sequence 61-171 of COPV L2 was tested. A composition was provided in a free form, nonconjugated to TMV (PV-L2₆₁₋₁₇₁), or as part of a TMV-coat fusion protein, conjugated to TMV (PV-L2₆₁₋₁₇₁/TMV).

Test dogs received one of the test compositions (PV-L2₆₁₋₁₇₁ or PV-L2₆₁₋₁₇₁/TMV), and were then exposed to COPV. A first negative control dog received saline, and was then exposed to COPV. A second negative control dog received TMV alone, and was then exposed to COPV. A positive control dog received the L1 VLP composition, and was then exposed to COPV. The results of the study are set forth in Table 4.

TABLE 4

| | | | Antibody |
|---|---|---|---|
| | Composition/ | | Titer |
| Animal | Saline | Warts | Response |
| Negative | Saline | Warts on both sites (+/+) | 0 |
| Control Dog | | | |
| Negative | TMV | Warts on both sites (+/+) | 0 |
| Control Dog | | | |
| (TMV) | | | |
| Positive | L1 Composition | No warts (−/−) | 0 |
| Control Dog | | | |
| Test Dog 1 | PV-L2₆₁₋₁₇₁ | No warts (−/−) | High |
| Test Dog 2 | PV-L2₆₁₋₁₇₁ | No warts (−/−) | Mid |
| Test Dog 3 | PV-L2₆₁₋₁₇₁ | Warts on one site (+/−) | Mid |
| Test Dog 4 | PV-L2₆₁₋₁₇₁ | Warts on both sites (+/+) | Low |
| Test Dog 5 | PV-L2₆₁₋₁₇₁/ | No warts (−/−) | High |
| | TMV | | |
| Test Dog 6 | PV-L2₆₁₋₁₇₁/ | No warts (−/−) | High |
| | TMV | | |
| Test Dog 7 | PV-L2₆₁₋₁₇₁/ | Warts on both sites (+/+) | Low |
| | TMV | | |

The negative control dog receiving saline had warts on the buccal mucosa after the oral exposure to the virus. There were extensive warts on one inoculation site, and the other site had less developed warts. Similarly, the negative control dog receiving TMV had 9 warts on one site and beginning of extensive warts on the other site. The positive control dog did not develop warts on either inoculation site.

Of the test dogs that received the composition (PV-L2₆₁₋₁₇₁) not conjugated to TMV, two developed no warts, one had warts one site, and one had warts on both sites. The test dog with warts on one site had 4 small buccal mucosa warts on one site, but no warts were observed at the other challenge site. The test dog with warts on both sites had 8 small warts on one site and 4 warts on the other site.

Of the test dogs that received the composition (PV-L2₆₁₋₁₇₁/TMV) conjugated to TMV, two developed no warts, and one had warts one site. The test dog with warts on both sites had 7 warts on one site and 4 warts on the other site.

Of the test dogs that received the composition (PV-L2₆₁₋₁₇₁) not conjugated to TMV, the two that developed no warts had good antibody titer responses, the one that had warts on one site had a good antibody titer response, and the one that had warts on both sites had a low antibody titer response. Of the test dogs that received the composition (PV-L2₆₁₋₁₇₁/TMV) conjugated to TMV, the two that developed no warts had good antibody titer responses, and the one that had warts on one site had a low antibody titer response.

The results of the study indicate that the PV-L2₆₁₋₁₇₁ compositions were at least partially protective when used as compositions to protect beagles against challenge by COPV.

### Example 4

### Plant-Produced PV L2 Polypeptide, COPV L2₅₋₂₆₀

The study described herein included a positive control dog inoculated with an L1 composition as described in Suzich, et al. 1995, and a negative control animal inoculated with saline or tobacco mosaic virus (TMV), alone. In the studies described herein, the positive control animals were wart free, and the negative control animals developed large, confluent oral warts on both of the buccal mucosa.

Tobacco plant-produced PV L2 polypeptide including amino acid sequence 5-260 of COPV L2 was tested. A composition was provided in a free form, nonconjugated to TMV (PV-L2₅₋₂₆₀, or as part of a TMV-coat fusion protein, conjugated to TMV (PV-L2₅₋₂₆₀/TMV).

Test dogs received one of the test compositions (PV-L2₅₋₂₆₀ or PV-L2₅₋₂₆₀/TMV), and were then exposed to COPV. A negative control dog received TMV alone, and was then exposed to COPV. A positive control dog received the L1 VLP composition, and was then exposed to COPV. The results of the study are set forth in Table 5.

TABLE 5

| | | | Antibody |
|---|---|---|---|
| | | | Titer |
| Animal | Composition/Saline | Warts | Response |
| Negative | TMV | Warts on both sites (+/+) | 0 |
| Control Dog | | | |
| Positive | L1 VLP | No warts (−/−) | 0 |
| Control Dog | Composition | | |
| Test Dog 1 | PV-L2₅₋₂₆₀ | No warts (−/−) | High |
| Test Dog 2 | PV-L2₅₋₂₆₀ | No warts (−/−) | High |
| Test Dog 3 | PV-L2₅₋₂₆₀ | No warts (−/−) | High |
| Test Dog 4 | PV-L2₅₋₂₆₀ | No warts (−/−) | Mid |
| Test Dog 5 | PV-L2₅₋₂₆₀ | No warts (−/−) | Low |
| Test Dog 6 | PV-L2₅₋₂₆₀/TMV | No warts (−/−) | High |
| Test Dog 7 | PV-L2₅₋₂₆₀/TMV | No warts (−/−) | Low |
| Test Dog 8 | PV-L2₅₋₂₆₀/TMV | Warts on one site (+/−) | Low |
| Test Dog 9 | PV-L2₅₋₂₆₀/TMV | Warts on both sites (+/+) | Low |
| Test Dog 10 | PV-L2₅₋₂₆₀/TMV | Warts on both sites (+/+) | Low |

The negative control dog receiving TMV had warts on the buccal mucosa after the oral exposure to the virus. The positive control dog did not develop warts.

Of the test dogs that received the composition (PV-L2₅₋₂₆₀ not conjugated to TMV, none developed warts, and each had an antibody titer response, four had good antibody titer responses, and one had a low antibody titer response.

Of the test dogs that received the composition (PV-L2₅₋₂₆₀/TMV) conjugated to TMV, two developed no warts and had good or low antibody titer responses, one had warts on one site and had a low antibody titer response, and two had warts on both sites and a low antibody titer response.

The results of the study indicate that the PV-L2₅₋₂₆₀/TMV composition is at least partially protective, while the free (non-conjugated to TMV) PV-L2₅₋₂₆₀ composition is fully protective.

### Example 5

### PV L2 Polypeptides Produced Using Eukaryotic Expression Systems

Construction and screening of Streptavidin—COPV/HPV L2 fragment fusions. A fusion protein was constructed including streptavidin and a COPV L2 polypeptide consisting of the amino acids 5-260 of COPV L2 (COPV L2₅₋₂₆₀) and employing tobacco-optimized codon usage. A 6-histidine tag was also included to permit purification by metal affinity chromatography. The COPV L2₅₋₂₆₀ fragment was tested as both an N (ID 1858) and C (ID 1861) terminal fusion to SA. To evaluate expression of both constructs, RNA transcripts were generated using the MMESSAGE MMACHINE™ T7 transcription kit (Ambion, Austin, Tex., U.S.A.) and inoculated onto 22 day old N. benthamiana plants. Following inoculation, the plants exhibited the typical mosaic phenotype on the systemically-infected tissue. Expression and solubility of these constructs was evaluated by a small-scale extraction under acidic (50 mM sodium acetate, pH 5, 3:1 buffer:tissue ratio) and alkaline conditions (50 mM Tris, pH 8, 3:1 buffer:tissue ratio) and analyzed by western blot. Expression was similar for both constructs, but notably reduced relative to the ID 1825 construct (for COPV-L2₆₁₋₁₇₁-SA). Under acidic conditions neither construct was soluble. This was also the case for the N-terminal fusion ID 1858 when extracted under alkaline conditions, however, for ID 1861 soluble product was recovered with the Tris buffer. Optimization of a purification method for pID 1861 was therefore initiated in parallel with the development of other COPV and HPV fusions.

To determine if a shorter COPV sequence would express at a higher level, four additional SA fusions for COPV were constructed, corresponding to amino acids 11-130 of the L2 protein (COPV₁₁₋₁₃₀). These included both N-terminal and C-terminal fusions to SA, with and without a 6-histidine tag. Additionally, HPV fusions homologous to the new COPV fusions, employing an As Different As Possible (ADAP) codon usage, were also cloned. The composition of all 10 constructs is summarized in Table 6.

TABLE 6
Summary of clones generated, relative expression level and solubility
(recovery in the S1 or S1 PEI) based on small-scale screening.

| pID # | Composition | GJ | S1 | S1 PEI |
|---|---|---|---|---|
| 1858 | His-COPV L2₅₋₂₆₀-SA | ++ | − | N/A |
| 1861 | SA-COPV L2₅₋₂₆₀-His | ++ | ++ | N/A |
| 3532 | SA-COPV L2₁₁₋₁₃₀ | ++ | ++ | ++ |
| 3533 | SA-HPV L2₁₁₋₁₃₀ | ++ | ++ | ++ |
| 3534 | COPV L2₁₁₋₁₃₀-SA | ++ | − | − |
| 3535 | HPV L2₁₁₋₁₃₀-SA | ++ | + | −/+ |
| 3536 | His-COPV L2₁₁₋₁₃₀-SA | ++ | − | − |
| 3537 | His-HPV L2₁₁₋₁₃₀-SA | ++ | + | −/+ |
| 3538 | SA-COPV L2₁₁₋₁₃₀-His | ++ | + | + |
| 3539 | SA-HPV L2₁₁₋₁₃₀-His | ++ | + | + |

N/A; not available.

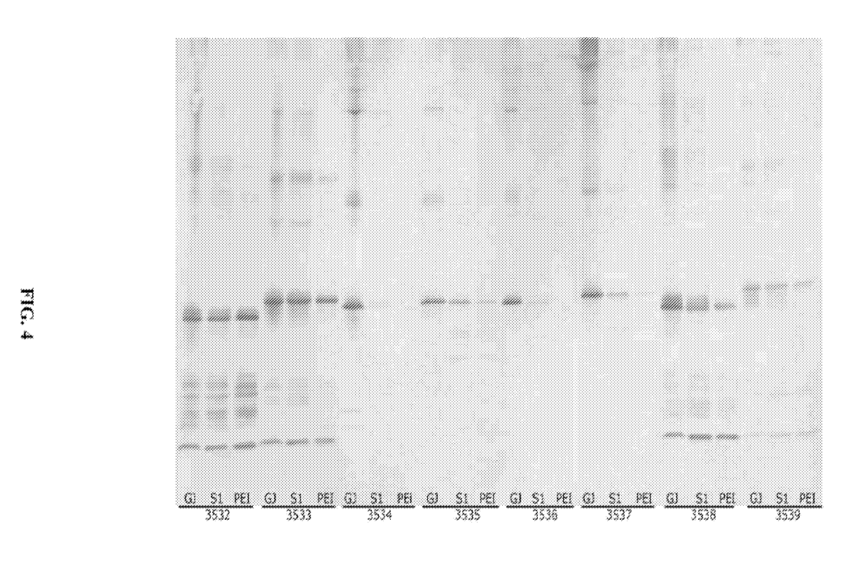

Similar to the pID 1861 and pID 1858 constructs, a small-scale screening was performed to assess expression level and solubility, extracting in 25 mM Tris, 10 mM EDTA pH 7.3 and using a 3:1 buffer:tissue ratio. The green juice (GJ) was adjusted from pH 6.8 to pH 7.5 and centrifuged at 10,000×g for 10 minutes, yielding the S1 supernatant. In parallel, a 1 ml sample of GJ was removed prior to pH adjustment and treated with 0.4% v/v polyethylenimine (PEI), to evaluate the extraction procedure employed for ID 1825 (COPV-L2₆₁₋₁₇₁-SA) purification. After a 20-minute incubation, the PEI-treated GJ was centrifuged at 6000×g for 5 minutes and the supernatant (S1 PEI) recovered. With reference to FIG. 4, the GJ, 51 and 51 PEI were analyzed by western blot. Briefly, the samples were run on SDS PAGE, blotted to nitrocellulose, probed with rabbit anti-streptavidin (Sigma, St. Louis, Mich.) and goat anti-rabbit IgG-alkaline phosphatase (Sigma) in combination with the BCIP/NBT kit (Invitrogen, Carlsbad, Calif., U.S.A.) were employed for detection. The relative expression levels and solubility (recovery in the S1 supernatant following centrifugation) are summarized in Table 6.

By western blot, the accumulation of product in the GJ was comparable for all constructs. Similar to the COPV L2₅₋₂₆₀ constructs, the solubility of the HPV/COPV L2₁₁₋₁₃₀ SA C-terminal constructs was better than the corresponding N-terminal constructs. In addition, the small-scale screening indicated that processing in the presence of PEI did not appear to reduce solubility. However, the level of truncation was greater for the C-terminal fusions. Based on the solubility data, the four C-terminal fusions (IDs 3532, 3533, 3538, 3539), along with ID 1861, were further evaluated to determine appropriate processing conditions for their purification. After a series of iterative optimization tests, two constructs were selected for manufacturing: pID 1861 for the COPV fusion, and pID 3533 for the HPV fusion.

SA-COPV L2₅₋₂₆₀-6 His extraction and purification. Initial screens of ID 1861 indicated that the fusion was soluble at alkaline pH, but precipitated under acidic conditions. Acidic pH treatment of the GJ is the standard clarification method employed as with centrifugation it efficiently removes both rubisco and pigment from the initial supernatant, allowing for efficient 0.45 um filtration prior to chromatography. Due to the observed insolubility, extraction under alkaline conditions was evaluated, however, the resulting S1 supernatant rapidly fouled 0.45 um filter units, preventing the efficient processing of plant tissue batches greater than 25 grams.

An alternative route for S1 clarification, compatible with extraction under alkaline conditions, is the addition of the polycationic polymer, PEI. Based on the protocol employed for purification of ID 1825 (COPV L2₆₁₋₁₇₁-SA) an initial extraction in the presence of 0.4% v/v PEI was tested. However, for pID 1861, the adjustment of the GJ to 0.4% PEI caused a majority of the fusion product to partition into the P1 pellet. Therefore, various PEI concentrations were tested for optimal clarification of the supernatant and rubisco removal, while maintaining the solubility of the fusion. With addition of 0.1% v/v PEI to the supernatant S1, a majority of the rubisco was precipitated and approximately 50% of the product remained soluble.

During ID 1825 recovery, the PEI-treated S1 supernatant is adjusted to 25% ammonium sulfate saturation, to selectively precipitate and partially purify the full-length SA fusion from truncation species prior to chromatography. At 25% saturation, the majority of the degraded SA tetramers remain soluble. For ID 1861, different saturation levels of ammonium sulfate were tested. Adjusting the supernatant to 20% saturation of ammonium sulfate appeared to efficiently precipitate the SA fusion. However, resolubilization of the pellet after ammonium sulfate precipitation proved to be difficult. By SDS PAGE and western blot analysis, a substantial amount of the resuspended fusion product was pelleted by centrifugation prior to 0.45 μm filtration.

Therefore an ID 1861 test extraction was performed, employing 0.1% PEI and no ammonium sulfate precipitation prior to chromatography, and the SA-fusion was subsequently purified by either immobilized metal affinity chromatography (IMAC) or iminobiotin affinity chromatography. With pH 4 elution, a peak was obtained from the iminobiotin resin, however, in the case of IMAC chromatography, no peak was observed with imidazole elution, as the PEI appeared to strip the nickel from the column. The pooled iminobiotin chromatography fractions were dialyzed against 1×PBS. By SDS PAGE, approximately 50% of the SA fusion was lost during dialysis. As a result passivation of the dialysis membrane prior to use was performed during subsequent processing, to reduce losses due to SA fusion adsorption. The final PBS-dialyzed SA fusion was stored at 4° C. and sampled periodically to assess stability. After 48 hours degradation was substantial.

IMAC was therefore evaluated as a polishing step after the initial purification with iminobiotin, in an attempt to remove the associated proteolytic activity and assess whether the low MW species could be separated from the full length SA fusion. For the IMAC peak fractions, a 30% ammonium sulfate precipitation was performed to concentrate the SA fusion. In contrast to ammonium sulfate precipitation from the 51 supernatant, resolubilization of the purified product was successful. Subsequent testing showed improved stability at 4° C., but indicated that the final composition should be stored at −20° C. in order to prevent proteolysis. With regard to the truncation bands, no change in the SA fusion profile was observed following IMAC, indicating that the tetramers were heterogeneous in nature.

Based on the above testing, the following final protocol was employed to isolate ID 1861. Systemically infected tissue was homogenized in 3 volumes of extraction buffer (50 mM Tris, 10 mM EDTA, 0.04% w/v sodium metabisulfite) in a Waring blender. The homogenate was strained through cheesecloth, adjusted to pH 7.2-7.8 and centrifuged at 10,000×g for 10 minutes to obtain the S1 supernatant. PEI was added to the S1 (0.1% v/v final) and following incubation at 4° C. for 20 minutes, the sample was centrifuged at 10,000×g for 10 minutes. The S1 PEI supernatant was adjusted to pH 10.5 and the non-proteinaceous precipitate that formed was removed by centrifugation (10,000×g for 10 minutes). This clarified supernatant was sequentially filtered through 1.0 um glass fiber and 0.45 um filters prior to iminobiotin chromatography. After chromatography, the peak fractions were combined, adjusted to pH 9, and dialyzed into 1×PBS using Tween-20 passivated dialysis tubing. The dialyzed SA fusion was then adjusted to 10 mM imidazole in preparation for polishing by IMAC chromatography. The peak fractions eluted from the IMAC resin were combined and adjusted to 30% saturation with ammonium sulfate to precipitate and concentrate the SA fusion. After 2 hours on ice, the sample was centrifuged at 10,000×g for 10 minutes. The SA fusion pellet was resuspended in 1×PBS and centrifuged at 20,000×g for 10 minutes to remove any insoluble product. The final supernatant was 0.2 um sterile filtered and stored at −20° C.

To minimize endotoxin contamination, glassware was rinsed and baked, plasticware was bleached and autoclaved, and buffers were prepared using water for irrigation (WFI) and 0.2 um sterile filtered.

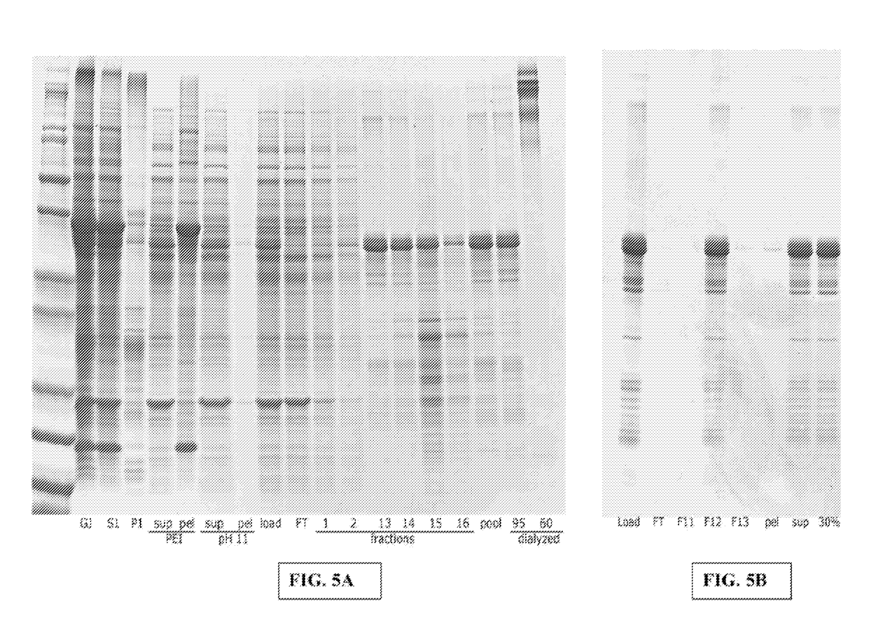

A representative gel for the optimized ID 1861 processing is shown in FIGS. 5A and 5B. Under the alkaline conditions employed, the SA fusion partitions principally into the S1 supernatant and the rubisco was effectively precipitated by 0.1% v/v PEI addition. No losses occurred with the adjustment to pH 11 or with the filtration prior to chromatography. Based on the flowthrough (FT), all of the SA fusion was captured by the iminobiotin resin and was recovered with elution at pH 4. Comparing the pooled and dialyzed samples, no losses occurred with dialysis using Tween-20 passivated tubing. The full length SA fusion (migrating at 45-50 kDa) was the principal product, and a series of truncated bands were present. Together with the full-length product all of the lower MW bands shift to 150-200 kDa with heating to 60° C., indicating that the truncation products retain the ability to form tetramers and suggesting that the tetramers are likely heterogeneous. Further purification employing IMAC (FIG. 5B) supports the latter hypothesis, as by SDS PAGE the peak eluted fraction profile was identical to that of the load. Precipitation with 30% ammonium sulfate resulted in recovery of the SA fusion as a soluble product.

The production runs and product recoveries are summarized in Table 7. The two lots were combined prior to vaccine manufacture for a total of 31.8 mg, which translates to a purified product yield of approximately 138 mg/kg tissue.

TABLE 7
Production runs and product recoveries for ID 1861.

| | | Purified 1861 | Purified 1861 | Purified 1861 |
|---|---|---|---|---|
| | | SA fusion | SA fusion | SA fusion |
| Lot | g tissue | mg/ml | ml available | mg total |
| Lot 1861-1 | 114 | 1.4 | 12 | 16.8 |
| Lot 1861-2 | 115 | 1.5 | 10 | 15 |

SA-HPV L2₁₁₋₁₃₀ extraction and purification. For the initial small-scale screening of the SA-HPV L2₁₁₋₁₃₀ (ID 3533) construct, addition of 0.4% v/v PEI did not appear to affect the solubility of the fusion; however, the screening did not continue past the S1 PEI stage. To test this method at scale, 25 grams of tissue was homogenized in 3 volumes of buffer (25 mM Tris-maleic acid, pH 7.3, with 0.04% sodium metabisulfite) in a Waring blender and the green juice (GJ) was adjusted to 0.4% (v/v) PEI. After 20 minutes at 4° C., the supernatant (S1) was obtained by centrifugation at 6000×g for 5 minutes and adjusted to 25% saturation with ammonium sulfate. After 2 hours on ice the samples were centrifuged at 10,000×g for 15 minutes. The pellets were resuspended in 25 mM ammonium carbonate, 0.5 M NaCl, pH 8.5 buffer and chromatography was performed with an immobilized iminobiotin column. A weak 32-kDa band was observed by SDS PAGE; however, a majority of the product was lost during the 0.45 um filtration step, indicating poor resolubilization of the fusion after the ammonium sulfate precipitation, similar to the 1861 SA fusion. Therefore, the ammonium sulfate precipitation step, to concentrate the fusion from the S1, prior to chromatography was omitted.

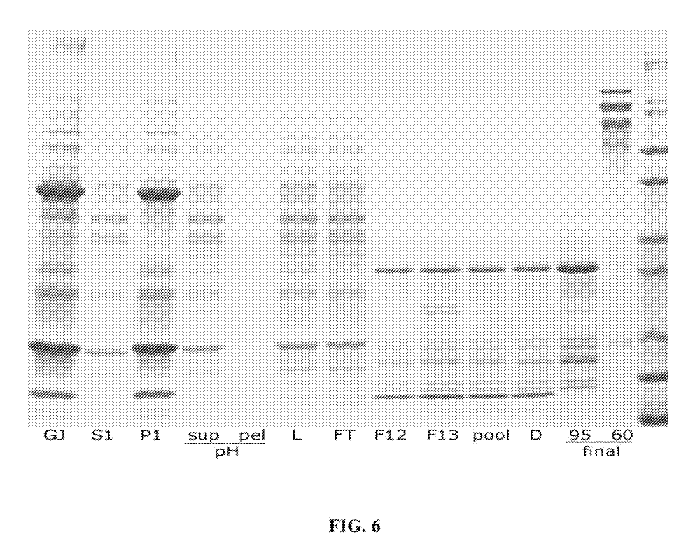

Next a series of small-scale extractions were performed to test the amount of PEI for optimal clarity and both the S1 supernatant and initial (P1) pellet were analyzed to assess the partitioning of the fusion. A minimum of 0.1% v/v PEI was required for rubisco precipitation from the S1, and at this concentration, approximately 50% of the fusion remained insoluble. Higher PEI concentrations did not improve supernatant clarity or host protein precipitation and SA fusion recovery in the S1 supernatant was also lowered. A third purification of pID 3533 was tested (20 g tissue), employing 0.1% PEI and omitting the ammonium sulfate precipitation prior to chromatography. From the SDS-PAGE and western blot, the majority (60-80%) of the SA fusion partitioned into the P1, but a Coomassie-stainable band was visible in the S1 (FIG. 6). By this method, SDS-PAGE indicated that the peak fractions contained both the putative full-length fusion (˜60%) and a prominent truncation products (˜40%) migrating at ˜12 kDa, the molecular weight for the SA core alone. A polishing ammonium sulfate precipitation step was effective at separating the putative full-length species from the 12 kDa truncation product with acceptable recoveries. In contrast to ammonium sulfate precipitation from the S1 supernatant, resolubilization of the purified product was successful. Although recovery for the 3533 SA fusion was suboptimal, the following processing method was employed for the manufacture of the SA-HPV L2₁₁₋₁₃₀ fusion, as sufficient product could be recovered to permit an initial evaluation of immunogenicity in vivo.

Briefly, tissue was homogenized in 3 volumes of extraction buffer (25 mM Tris-maleic acid, pH 7.5, 0.04% w/v sodium metabisulfite) in a Waring blender. The homogenate was passed through cheesecloth and the GJ adjusted to 0.1% PEI. After a 20 minute incubation on ice, the sample was centrifuged at 6000×g for 5 minutes to obtain a supernatant S1. The S1 was adjusted to pH 10.5 and centrifuged at 10,000×g for 10 minutes to remove precipitate. This clarified supernatant was filtered through a 1 um glass fiber prefilter and a 0.45 um filter. Following iminobiotin chromatography, the peak fractions were pooled, adjusted to pH 9, and dialyzed against 1×PBS. The dialyzed material was adjusted to 30% saturation of ammonium sulfate and incubated on ice for 2 hours. The sample was centrifuged at 20,000×g for 15 minutes, and the resulting pellet resuspended in 1×PBS. A second centrifugation for 5 minutes to remove any insoluble product was performed, and the supernatant was stored at −20° C.

The production runs and product recoveries for ID 3533 are summarized in Table 8. The four lots were combined prior to vaccine manufacture for a total of 29.5 mg from approximately 1 kg of processed tissue.

TABLE 8
Production runs and recoveries for ID 3533.

| | | Purified 3533 | Purified 3533 | Purified 3533 |
|---|---|---|---|---|
| | | SA fusion | SA fusion | SA fusion |
| Lot | g tissue | mg/ml | ml available | mg total |
| 3533-1 | 196 | 1.1 | 3 | 3.3 |
| Lot 3533-2 | 270 | 1.37 | 10 | 13.7 |
| Lot 3533-3 | 270 | 1.35 | 6 | 8.1 |
| Lot 3533-4 | 270 | 1.1 | 4 | 4.4 |

Complex formation and vaccine preparation. Prior to complex formation, the TMV was treated with 5 mM binary ethyleneimine (BEI) at 37° C. for 48 hours to inactivate the virus and for sterilization. Excess BEI was neutralized by the addition of a 3-fold molar excess of sodium thiosulfate, followed by pH adjustment and dilution. Because the COPV fusion degraded at 37° C., sterilization by BEI treatment was not an option. Therefore, the COPV L2 and HPV L2 fusions were 0.2 um sterile filtered prior to vialing or loading onto TMV.

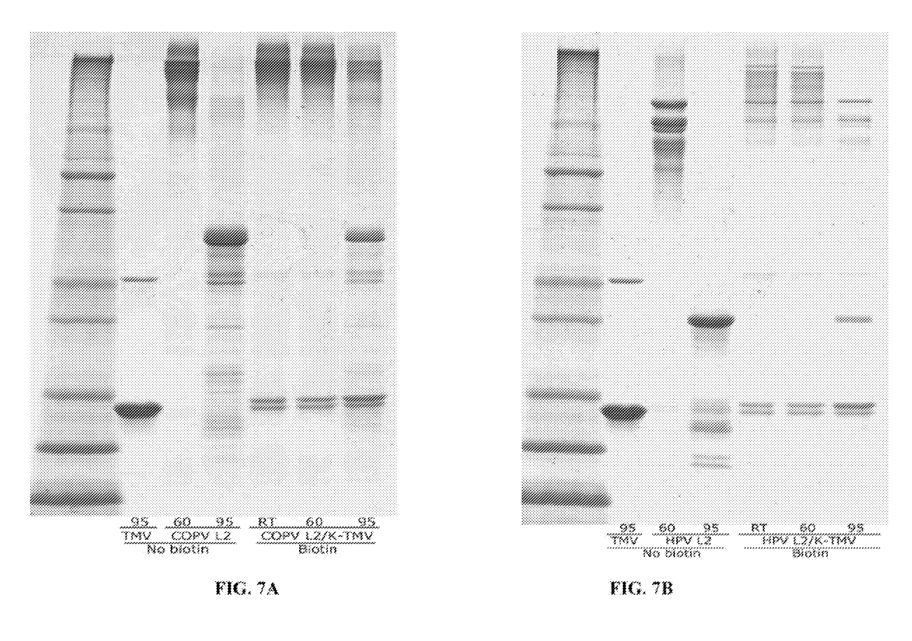

For complex loading, the fusion and the biotinylated ID 1295.4 virions were combined in a 1:1 molar ratio and incubated for 3 hours at room temperature, a target loading of 25%. This translates to approximately 555 tetramers of SA-COPV L2₅₋₂₆₀-6 His (2220 SA-COPV L2₅₋₂₆₀-6 His fragments) or 548 tetramers of SA-HPV L2₁₁₋₁₃₀ (2192 SA-HPV L2₁₁₋₁₃₀ fragments) per capsid. The loading of the fusions onto biotinylated ID 1295.4 (K-TMV) was characterized by SDS PAGE band shift analysis (FIG. 7). Because the detergent SDS causes the TMV capsid to disassociate, even in the absence of heating, a 5-fold molar excess of biotin was added to the complex samples prior to the addition of SDS PAGE loading dye. This addition of biotin saturates any unoccupied biotin binding sites, thereby preventing additional association of tetramer/biotinylated coat protein from occurring and allows for a more accurate analysis. However, when biotin is present in molar excess, stability is increased, with a reduction in monomer dissociation occurring with heating. Due to their heterogeneous composition, the tetramers run on the gel as a series of diffuse bands rather than one distinct band.

The vialed antigen lot numbers, the release specifications and the results for each antigen are summarized in Tables 9 and 10 below. All antigens conformed to all release specifications.

TABLE 9
Release specifications and product certification for the manufactured COPV L2 and
control antigens.

| | Antigen | | |
|---|---|---|---|
| | | | COPV L2/K-TMV |
| | K-TMV | COPV L2 | 0.2 um sterile filtered/BEI |
| | BEI treated | 0.2 um sterile filtered | treated |
| | Lot No. | | |
| | 1295.426MAY06CP | 186126MAY06L2 | 186126MAY06L2CP |
| Concentration (BCA) | 0.33 mg/ml | 0.78 mg/ml | 1.1 mg/ml |
| Appearance | opalescent, colorless, clear | opalescent, colorless, clear | opalescent, colorless, clear |
| % Full length, by | 100% | 79.1%** | K-TMV - 100% |
| SDS PAGE | | | COPV L2 - 79.1% |
| pH | 7.30 | 7.32 | 7.33 |
| Bioburden Assay* | passed | passed | passed |
| Storage Buffer | PBS | PBS | PBS |

*The sample passes this assay when no colonies are detected on growth media incubated at room temperature for 4 days, followed by 3 additional days at 37 C.
**Balance represents truncated streptavidin fusion protein that retains the ability to form tetramers.

TABLE 10
Release specification and product certification for the manufactured HPV L2 and
control antigens.

| | Antigen | | |
|---|---|---|---|
| | | | HPV L2/K-TMV |
| | K-TMV | HPV L2 | 0.2 um sterile filtered/BEI |
| | BEI treated | 0.2 um sterile filtered | treated |
| | Lot No. | | |
| | 1295.426MAY06CP-2 | 353326MAY06L2 | 353326MAY06L2CP |
| Concentration (BCA) | 0.57 mg/ml | 0.84 mg/ml | 1.41 mg/ml |
| Appearance | opalescent, colorless, clear | opalescent, colorless, clear | opalescent, colorless, clear |
| Purity, by SDS PAGE | 100% | 72.6%** | K-TMV - 100% |
| | | | HPV L2 - 72.6% |
| pH | 7.39 | 7.22 | 7.25 |
| Bioburden Assay* | passed | passed | passed |
| Storage Buffer | PBS | PBS | PBS |
| Storage conditions | −20 C. | −20 C. | −20 C. |

*The sample passes this assay when no colonies are detected on growth media incubated at room temperature for 4 days, followed by 3 additional days at 37 C.
**Balance represents truncated streptavidin fusion protein that retains the ability to form tetramers.

### Example 6

### Purification of the Papillomavirus L2 Protein from Eukaryotic Systems

To evaluate the factors important to the purification of papillomavirus L2 fusion proteins in eukaryotic systems a series of constructs were designed as outlined in Table 12. In these constructs, which employed the streptavidin core (SA) as a fusion partner, the following factors were varied to evaluate the impact on fusion protein purification characteristics:
Length of Papillomavirus L2 Protein fused to Streptavidin;
Papillomavirus type: Human papillomavirus (HPV) and canine oral papillomavirus (COPV);
Fusion location relative to the streptavidin core (N-terminal vs. C-terminal); and
Presence of 6 histidine metal affinity tag.

For the purposes of this example, the papillomavirus L2 fusions to streptavidin are denoted as L2-SA, irrespective of relative position of the two fusion protein components and the numeric identifier (Table 11) used when a specific construct is under consideration.

TABLE 11
Summary of clones generated to evaluate
factors impacting Papillomavirus L2 fusion protein
purification characteristics.

| Identifier | Structure |
|---|---|
| 1825 | COPV-L2₆₁₋₁₇₁-SA |
| 1854 | 6 His-COPV L2₆₁₋₁₇₁-SA |
| 1858 | 6 His-COPV L2₅₋₂₆₀-SA |
| 1861 | SA-COPV L2₅₋₂₆₀-6 His |
| 3532 | SA-COPV L2₁₁₋₁₃₀ |
| 3533 | SA-HPV L2₁₁₋₁₃₀ |
| 3534 | COPV L2₁₁₋₁₃₀-SA |
| 3535 | HPV L2₁₁₋₁₃₀-SA |
| 3536 | 6 His-COPV L2₁₁₋₁₃₀-SA |
| 3537 | 6 His-HPV L2₁₁₋₁₃₀-SA |
| 3538 | SA-COPV L2₁₁₋₁₃₀-6 His |
| 3539 | SA-HPV L2₁₁₋₁₃₀-6 His |

The extraction of recombinant proteins from plant-based systems typically consists of an extraction in 1-3 volumes of water and adjustment to a pH of approximately 5. Alternatively a buffer can be used to provide for a final pH of approximately 5. Under these acidic conditions, the majority of the host proteins in the green juice extract (GJ) aggregate and can be separated from the recombinant protein of interest by centrifugation. The resultant pellet (hereafter denoted P1) is discarded and the supernatant (hereafter denoted S1) is carried forward for further processing. However, this generally employed procedure was not applicable to the L2-SA fusions, which partitioned predominantly into the P1 pellet under acidic conditions and could not be subsequently extracted. In addition degradation of the L2-SA fusion was generally greater at acidic pH (pH 5) necessitating the use of neutral or alkaline conditions, which reduced the level of observed proteolysis.

Initial testing focused on 1825 L2-SA which consists of a 112 amino acid domain from the L2 protein of canine oral papillomavirus (COPV), corresponding to amino acids 61-171, fused to the N terminus of streptavidin. Extractions were performed at alkaline pH (pH 7.5-8.0) under high (100 mM phosphate buffer) or low (25 mM Tris/Maleic acid buffer) ionic strength conditions. Under these conditions significant levels of host protein were present in the S1 supernatant, hindering preparation of an extract amenable to chromatography. Inclusion of the polyethylenimine (PEI), an aliphatic polyamine polymer of high molecular weight and very high cationic charge was tested as a means to improve host protein precipitation. Concentrations of PEI in the range 0.01% w/v to 0.4% w/v were evaluated and the effectiveness at the selective precipitation of contaminating proteins evaluated. At concentrations of 0.1% w/v PEI removal of the green pigment from the S1 was obtained at both ionic strengths, while contaminating protein precipitation was found to be strongly dependent on ionic strength. Under low ionic strength the principal host protein (rubisco) as well as the expression vector derived tobacco mosaic virus (TMV) coat protein were effectively partitioned to the P1, with the majority of the 1825 L2-SA remaining soluble. In contrast, minimal contaminating protein precipitation was obtained with 0.4% w/v PEI when 100 mM phosphate buffer was employed.

Ammonium sulfate precipitation was used to isolate the 1825 L2-SA protein from the PEI and concentrate prior to chromatography. Up to 50% ammonium sulfate was tested and 25-30% found to be optimal. With 25-30% ammonium sulfate the full length 1825 L2-SA precipitated from solution while truncated species and remaining host protein were soluble. The resuspended ammonium sulfate pellet, consisting of approximately 70% 1825 L2-SA, was further purified by iminobiotin affinity chromatography.

The final optimizes process for the 1825 L2-SA construct was the following. Plant tissue was homogenized in 3 volumes of extraction buffer (25 mM Tris-maleic acid buffer, pH 7.5, 0.01% w/v sodium metabisulfite). Following addition of 0.4% (w/v) polyethyleneimine (PEI), the homogenate was centrifuged, a 30% ammonium sulfate cut was performed on the clarified S1 supernatant, and the resulting pellet was resuspended. The clarified S1 supernatant was adjusted to pH 11 and loaded onto an immobilized iminobiotin column (Pierce, Rockford, Ill.). The fusion was eluted with 0.1 M acetic acid, pH 4.0, and the peak fractions were concentrated with a 50% ammonium sulfate cut, followed by resuspension in phosphate-buffered saline, pH 7.4 (Invitrogen, Carlsbad, Calif.).

The subsequent constructs listed in Table 11 evaluated inclusion of a 6 Histidine tag, impact of L2 protein fragment size and source, as well as fusion location. The fusion of the L2 protein to the C-terminus versus the N-terminus of streptavidin was found to alter partitioning into the P1 pellet and S1 supernatant during the initial separation. For the 11-130 L2 fusions, accumulation (level in the initial extract as determined by Western blot) was comparable across constructs. However, the C-terminal fusions generally showed greater solubility, and partitioning to the S1 supernatant was generally higher for the non-His tagged constructs. From a product recovery standpoint a non-his tagged C-terminal fusion to streptavidin therefore may be more favorable for the papillomavirus L2 protein. For the 5-260 L2 fusions the C-terminal construct (1861 L2-SA) was also the more soluble, consistent with the 11-130 L2 fusion observation. Note that this comparison was not performed for 1825 or 1854 L2-SA, which were both determined to have acceptable solubility, as no C-terminal fusions to streptavidin were prepared in these two cases.

For the 11-130 L2 fusions, 3533 L2-SA was carried forward for evaluation and the extraction performed initially per the protocol developed for the 1825 L2-SA construct. PEI at 0.4% w/v was used for host protein partitioning into the P1 pellet and the S1 supernatant was adjusted to 25% saturation with ammonium sulfate. However, analysis by western blot indicated that in contrast to the 1825 L2-SA construct, substantial product partitioning to the P1 pellet was obtained and resolubilization of the fusion precipitated from the S1 supernatant using ammonium sulfate was suboptimal. As a result testing was performed to optimize PEI concentration and the ammonium sulfate precipitation step was omitted prior to chromatography. With 0.1% v/v PEI effective rubisco precipitation from the S1 was obtained and approximately 50% of the fusion remained in soluble. The product recovered from iminobiotin affinity chromatography contained both the full-length fusion (˜60%) and a prominent truncation products (˜40%) migrating at 12 kDa, the molecular weight for the SA core alone. A polishing ammonium sulfate precipitation step was effective at separating the full-length species from the 12 kDa truncation product with acceptable recoveries. In contrast to ammonium sulfate precipitation from the S1 supernatant, resolubilization of the purified product was successful.

The finalized process for the 3533 L2-SA fusion was the following. Infected tissue was homogenized in 3 volumes of extraction buffer (25 mM Tris-maleic acid, pH 7.5, 0.04% w/v sodium metabisulfite) in a Waring blender. The homogenate was passed through cheesecloth and the GJ adjusted to 0.1% w/v PEI. After a 20 minute incubation on ice, the sample was centrifuged at 6000×g for 5 minutes to obtain a supernatant S1. The S1 was adjusted to pH 10.5 and centrifuged at 10,000×g for 10 minutes to remove precipitate. This clarified supernatant was filtered through a 1 um glass fiber prefilter and a 0.45 um filter. Following iminobiotin chromatography, the peak fractions were pooled, adjusted to pH 9, and dialyzed against 1×PBS. The dialyzed material was adjusted to 30% saturation of ammonium sulfate and incubated on ice for 2 hours. The sample was centrifuged at 20,000×g for 15 minutes, and the resulting pellet resuspended in 1×PBS.

As another illustration of the methods applicable to L2-SA fusion isolation from eukaryotic systems the 5-260 COPV L2 fusion (1861 L2-SA) was considered. The protocol developed for 1825 L2-SA was used as the starting point. However, for 1861 L2-SA adjustment of the GJ to 0.4% PEI caused a majority of the fusion product to partition into the P1 pellet. Therefore, various PEI concentrations were tested for optimal clarification of the supernatant and rubisco removal, while maintaining the solubility of the fusion. With addition of 0.1% v/v PEI to the supernatant S1, a majority of the rubisco was precipitated and 50-60% of the product remained soluble. To concentrate 1861 L2-SA different saturation levels of ammonium sulfate were tested. Adjusting the supernatant to 20% saturation of ammonium sulfate appeared to efficiently precipitate the SA fusion. However, resolubilization of the pellet after ammonium sulfate precipitation proved to be difficult, similar to the observation for 3533 L2-SA and this concentration step was omitted prior to iminobiotin chromatography. The recovered 1861 L2-SA was unstable, showing proteolytic degradation with storage. The presence of the 6-His tag allowed for metal affinity chromatography to be used as a polishing step after the initial purification with iminobiotin and a 30% ammonium sulfate precipitation was performed to concentrate the SA fusion. In contrast to ammonium sulfate precipitation from the S1 supernatant, resolubilization of the purified product was successful. Subsequent testing showed improved stability.

The finalized process for the 1861 L2-SA fusion was the following. Infected tissue was homogenized in 3 volumes of extraction buffer (50 mM Tris, 10 mM EDTA, 0.04% w/v sodium metabisulfite). The homogenate was adjusted to pH 7.2-7.8, followed by centrifugation at 10,000×g for 10 minutes. 0.1% v/v PEI was added to the supernatant and centrifugation was repeated. The resulting supernatant was adjusted to pH 10.5 and the non-proteinaceous precipitate that formed was removed by centrifugation at 10,000×g for 10 minutes. The clarified supernatant was sequentially filtered through 1.0 um glass fiber and 0.45 um filters prior to iminobiotin chromatography. After chromatography, the peak fractions were combined, adjusted to pH 9, and dialyzed into 1×PBS using Tween-20 passivated dialysis tubing, to minimize loss due to non-specific adsorption. The dialyzed fusion was then adjusted to 10 mM imidazole in preparation for polishing with IMAC chromatography (Amersham). The peak fractions were combined, concentrated by a 30% ammonium sulfate cut and the pellet was resuspended in 1×PBS.

### Example 7

### Production of a Novel HPV L2-Streptavidin Fusion Protein

A synthetic cDNA that encodes a fusion between the HPV-16 L2 amino terminal peptide sequence that encompasses amino acids 13 through 92, inclusive, was fused at the amino terminus of the streptavidin core protein. The synthetic cDNA and encoded polypeptide sequences (L2-SAUoL; SEQ ID NOs: 14-16) are shown below. The synthetic gene was cloned into a pUC-based plasmid. The L2-SAUoL synthetic DNA was excised using PacI and XhoI restriction endonucleases and cloned into a TMV-based GENEWARE® expression vector. Infectious RNA was produced by in vitro transcription of the viral cDNA using T7 RNA polymerase and reagents supplied with the MMESSAGE MMACHINE™ kit. The synthetic RNA was used to infect 22 day old Nicotiana benthamiana seedlings. After 8 days the N. benthamiana plants showed symptoms typical of TMV infection. Leaf extracts were prepared by grinding tissue in 0.2 M sodium acetate buffer, pH 4.0, with 250 mM NaCl. Protein extracts were separated by SDS-polyacrylamide gel electrophoresis. Western blots of separated proteins were probed with a rabbit polyclonal serum raised against HPV-16 L2 amino acids 11-200 (supplied by Richard Roden, Johns Hopkins University). A band that reacted with the L2 antiserum was clearly visible on western blots, which demonstrated that the L2-SAUoL product accumulates in infected plants.

### L2-SAUoL Sequences

cDNA Sequence

(SEQ ID NO: 14)

ttaattaaccatgGCCAGCGCCACCCAGCTGTACAAGACCTGCAAGCAGG
CCGGCACCTGCCCCCCCGACATCATCCCCAAGGTGGAGGGCAAGACCATC
GCCGACCAGATCCTGCAGTACGGCAGCATGGGCGTGTTCTTCGGCGGCCT
GGGCATCGGCACCGGCAGCGGCACCGGCGGCAGGACCGGCTACATCCCCC
TGGGCACCAGGCCCCCCACCGCCACCGACACCCTGGCCCCCGTGAGGCCC
CCCgcCGGCGGTGGAGGATCTGGTGGTGGTGGTTCTGGTGGAGGTGGAAG
TTCTGGAATTACTGGAACTTGGTACAACCAGCTTGGATCTACTTTCATTG
TGACTGCTGGAGCTGATGGTGCTCTTACTGGTACTTACGAGTCTGCTGTT
GGAAATGCTGAGTCAAGATACGTGTTGACAGGAAGATACGATTCTGCTCC
AGCTACTGATGGATCTGGAACTGCTCTTGGATGGACTGTTGCTTGGAAGA
ACAACTACAGGAACGCTCACTCTGCTACTACTTGGAGTGGACAGTATGTT
GGAGGAGCTGAGGCTAGGATTAACACTCAGTGGCTTCTTACTTCTGGAAC
TACTGAGGCTAACGCTTGGAAGTCTACTCTTGTGGGACACGATACTTTCA
CTAAGGTGAAGCCATCTGCTGCTTCTGCCGGGTAGcctaggctcgag

### Aligned DNA (SEQ ID NO: 15) and Polypeptide (SEQ ID NO: 16) Sequences

Total amino acid number: 224, MW=22425
Max ORF starts at AA pos 1(may be DNA pos 1) for 224 AA(672 bases), MW=22425 Sequence of HPV 16 L2 13-92 is underlined. A (Gly4Ser)₃ flexible linker sequence (bold and italics)links the L2 sequence to the streptavidin (SA) core sequence at the amino terminus of the SA Core.

| | |
|---|---|
| | 10 20 30 40 50 60 |
| 1 | ATGGCCAGCGCCACCCAGCTGTACAAGACCTGCAAGCAGGCCGGCACCTGCCCCCCCGAC |
| 1 | M A S A T Q L Y K T C K Q A G T C P P D |
| | 70 80 90 100 110 120 |
| 61 | ATCATCCCCAAGGTGGAGGGCAAGACCATCGCCGACCAGATCCTGCAGTACGGCAGCATG |
| 21 | I I P K V E G K T I A D Q I L Q Y G S M |
| | 130 140 150 160 170 180 |
| 121 | GGCGTGTTCTTCGGCGGCCTGGGCATCGGCACCGGCAGCGGCACCGGCGGCAGGACCGGC |
| 41 | G V F F G G L G I G T G S G T G G R T G |
| | 190 200 210 220 230 240 |
| 181 | TACATCCCCCTGGGCACCAGGCCCCCCACCGCCACCGACACCCTGGCCCCCGTGAGGCCC |
| 61 | Y I P L G T R P P T A T D T L A P V R P |
| | 250 260 270 280 290 300 |
| 241 | CCCGCCGGCGGTGGAGGATCTGGTGGTGGTGGTTCTGGTGGAGGTGGAAGTTCTGGAATT |
| 81 | P [symbol] [symbol] [symbol] I |
| | 310 320 330 340 350 360 |
| 301 | ACTGGAACTTGGTACAACCAGCTTGGATCTACTTTCATTGTGACTGCTGGAGCTGATGGT |
| 101 | T G T W Y N Q L G S T F I V T A G A D G |
| | 370 380 390 400 410 420 |
| 361 | GCTCTTACTGGTACTTACGAGTCTGCTGTTGGAAATGCTGAGTCAAGATACGTGTTGACA |
| 121 | A L T G T Y F S A V G N A F S R Y V L T |
| | 430 440 450 460 470 480 |
| 421 | GGAAGATACGATTCTGCTCCAGCTACTGATGGATCTGGAACTGCTCTTGGATGGACTGTT |
| 141 | G R Y D S A P A T D G S G T A L G W T V |
| | 490 500 510 520 530 540 |
| 481 | GCTTGGAAGAACAACTACAGGAACGCTCACTCTGCTACTACTTGGAGTGGACAGTATGTT |
| 161 | A W K N N Y R N A H S A T T W S G Q Y V |
| | 550 560 570 580 590 600 |
| 541 | GGAGGAGCTGAGGCTAGGATTAACACTCAGTGGCTTCTTACTTCTGGAACTACTGAGGCT |
| 181 | G G A E A R I N T Q W L L T S G T T E A |
| | 610 620 630 640 650 660 |
| 601 | AACGCTTGGAAGTCTACTCTTGTGGGACACGATACTTTCACTAAGGTGAAGCCATCTGCT |
| 201 | N A W K S T L V G H D T F T K V K P S A |
| | 670 |
| 661 | GCTTCTGCCGGGTAG |
| 221 | A S A G* |

### Example 8

HPV Cross-Neutralization Using an HPV L2-Streptavidin Fusion Protein

Purified L2-SAUoL protein is formulated with alum salt-based adjuvant and used to vaccinate guinea pigs at doses that range from 1 ug/dose to 100 micrograms/dose. In animals that receive three doses of the vaccine, antibodies can be detected that neutralize HPV-16 pseudovirions as well as HPV-31 pseudovirions. This demonstrates that the L2-SAUoL vaccine can induce antibodies that cross-neutralize HPV strains in Species 9 of the genus Alphapapillomavirus. Additionally, sera from guinea pigs that are vaccinated with the L2-SAUoL vaccine can induce antibodies that neutralize HPV-19 and HPV-45 pseudovirions. This demonstrates that the L2-SAUoL vaccine can induce antibodies that neutralize viruses in different species within the Alphapapillomavirus genus. In addition, beagle dogs that are vaccinated three times with adjuvanted L2-SAUoL protein can be protected against mucosal challenge with canine oral papillomavirus, which demonstrates that the L2-SAUoL vaccine can be a pan-papillomavirus prophylactic vaccine.

### Example 9

### Eukaryotic Expression Compared to Prokaryotic Expression of PV L2 Polypeptides

Tobacco plant-produced PV L2 polypeptide fragments, as disclosed herein and elsewhere (e.g., Smith, et al. 2006. Virology) and PV L2 polypeptide produced in a recombinant prokaryotic system (e.g., E. Coli) are each administered into test animals (e.g., beagle dogs or guinea pigs) and results compared for ability to generate a protective immune response in test animals. The PV L2 polypeptides produced from each system can be in a free form (nonconjugated), as part of a TMV-coat fusion protein, conjugated to TMV (PV-L2/TMV), or conjugated to streptavidin (SA).

Test animals each receive one of the test compositions from each system (and optionally conjugated or non-conjugated variations of peptides from each system), and then are exposed to PV (e.g., COPV for dog test animals). A negative control dog can receive carrier alone and/or conjugate, and then can be exposed to PV. A positive control dog can receive an L1 VLP composition, such as the L1 composition as described in Suzich, et al. 1995, and then can be exposed to PV.

It is expected that the negative control animals will not show an enhanced immune response. It is expected that the positive control animals will show an enhanced immune response. Of the test animals that receive the composition produced in the eukaryotic system, it is expected that these animals will develop a more robust immune response directed against PV than the test animals inoculated with the L2 peptides produced in a prokaryotic expression system. The extent of immune response can be measured with a number of different established protocols, such as disclosed in Example 2, and including serum antibody analyses (e.g., by ELISA), virus/pseudovirus neutralization assays, and PV test animal challenge.

The results of this study can indicate that the L2 compositions produced in the tobacco expression system can produce a superior immune response against PV as compared to L2 compositions produced in the prokaryotic expression system.

### REFERENCES

Throughout this document, various references are cited. All such references are incorporated herein by reference, including the references set forth in the following list.
Bell, J. A., Sundberg, J. P., Ghim, S. J., Newsome, J., Jenson, A. B., & Schlegel, R. (1994) Pathobiology 62, 194-198.
Bendahmane m, Koo M, Karrer E, Beachy R N. Display of epitopes on the surface of tobacco mosaic virus: impact of charge and isoelectric point of the epitope on virus-host interactions. J. Mol. Biol. 1999; 290(1): 9-20.
Campo, M. S., et al., A peptide encoding a B-cell epitope from the N-terminus of the capsid protein L2 of bovine papillomavirus-4 prevents disease. Virology, 1997. 234(2): p. 261-6.
Campo, M. S., Vaccination against papillomavirus in cattle. Clin Dermatol, 1997. 15(2): p. 275-83.
Chackerian B, Lowy D R, Schiller J T. Conjugation of a self-antigen to papillomavirus-like particles allows for efficient induction of protective autoantibodies. J. Clin. Invest. 2001; 108(3): 415-423.
Chandrachud L M, Grindlay G J, McGarvie G M, et al. Vaccination of cattle with the N-terminus of L2 is necessary and sufficient for preventing infection by bovine papillomavirus-4. Virology 1995; 211:204-8.
Christensen N D, Kreider J W, Kan N C, DiAngelo S L. The open reading frame L2 of cottontail rabbit papillomavirus contains antibody-inducing neutralizing epitopes. Virology 1991; 181:572-9.
Coligan et al. Current Protocols in Immunology, John Wiley & Sons Inc. (1994).
Davidson E J, Boswell C M, Sehr P, et al. Immunological and clinical responses in women with vulval intraepithelial neoplasia vaccinated with a vaccinia virus encoding human papillomavirus 16/18 oncoproteins. Cancer Res 2003; 63:6032-41.
de Jong A, O'Neill T, Khan A Y, et al. Enhancement of human papillomavirus (HPV) type 16 E6 and E7-specific T-cell immunity in healthy volunteers through vaccination with TA-CIN, an HPV16 L2E7E6 fusion protein composition. Composition 2002; 20:3456-64.
de Villiers E. M., Fauquet C, Broker T R, Bernard H U., and zur Hausen H. 2004. Classification of papillomaviruses. Virology, 324(1): 17-27.
Delius H, Van Ranst M A, Jenson A B, zur Hausen H, Sundberg, J P. Canine oral papillomavirus genomic sequence: a unique 1.5-kb intervening sequence between the E2 and L2 open reading frames. Virology. 1994, 204:447-52.
Domingo, G. J., S. Orru, and R. N. Perham. 2001. Multiple display of peptides and proteins on a macromolecular scaffold derived from a multienzyme complex. J Mol Biol 305:259-67.
Embers M E, Budgeon L R, Pickel M, Christensen N D. Protective immunity to rabbit oral and cutaneous papillomaviruses by immunization with short peptides of L2, the minor capsid protein. J Virol 2002; 76: 9798-805.
Fiander A N, Tristram A J, Davidson E J, et al. Primeboost vaccination strategy in women with high-grade, noncervical anogenital intraepithelial neoplasia: clinical results from a multicenter phase II trial. Int J Gynecol Cancer 2006; 16:1075-81.
Fields et al. Synthetic polyelectrolytes as tumour inhibitors. Nature. 1960 Jun. 4; 186:778-80.
Gambhira R, Gravitt P E, Bossis I, Stern P L, Viscidi R P, Roden R B S. Vaccination of healthy volunteers with human papillomavirus type 16 12e7e6 fusion protein induces serum antibody that neutralizes across papillomavirus species. Cancer Res 2006; 66(23):11120-11124.
Gambhira R, Jagu S, Karanum B, Gravitt P E, Culp T D, Christensen N D, Roden, R B S. Protection of rabbits against challenge with rabbit papillomaviruses by immunization with the N-terminus of HPV16 minor capsid antigen L2. J. Virol. 2007.
Gambhira, R., et al., A protective and broadly cross-neutralizing epitope of human papillomavirus L2. J Virol, 2007. 81(24): p. 13927-31.
Gambhira, R., et al., Protection of rabbits against challenge with rabbit papillomaviruses by immunization with the N terminus of human papillomavirus type 16 minor capsid antigen L2. J Virol, 2007. 81(21): p. 11585-92.
Gambhira, R., et al., Vaccination of healthy volunteers with human papillomavirus type 16 L2E7E6 fusion protein induces serum antibody that neutralizes across papillomavirus species. Cancer Res, 2006. 66(23): p. 11120-4.
Gaukroger, J. M., et al., Vaccination of cattle with bovine papillomavirus type 4 L2 elicits the production of virus-neutralizing antibodies. J Gen Virol, 1996. 77 (Pt 7): p. 1577-83.
Ghim S., J. A. Suzich, J. Tamura, J. A. Bell, W. White, J. Newsome, F. Hill, P. Warrener, J. Sundberg, A. B. Jenson and R. Schlegel. Formalin-inactivated oral papilloma extracts and recombinant L1 compositions protect completely against mucosal papillomavirus infection: a canine model. Compositions 95, 375-379, 1995.
Ghim, S, Basu, P S, and Jenson, A B. Cervical cancer, etiology, pathogenesis treatment and future compositions. Asian Pacific Journal of Cancer Prevention 3 (3), 207-214, 2002.
Ghim, S., J. Newsome, J. Bell, J. P. Sundberg, R. Schlegel, and A. B. Jenson. 2000. Spontaneously regressing oral papillomas induce systemic antibodies that neutralize canine oral papillomavirus. Exp Mol Pathol 68:147-51.
Harper D M, Franco E L, Wheeler C, Ferris D G, et al. Efficacy of a bivalent L1 virus-like particle composition in prevention of infection with human papillomavirus types 16 and 18 in young women: a randomized controlled trial. Lancet 2004; 364 (9447): 1757-1765.
Harper D M, Franco E L, Wheeler C M, et al. Sustained efficacy up to 4.5 years of a bivalent L1 virus-like particle composition against human papillomavirus types 16 and 18: follow-up from a randomised control trial. Lancet 2006; 367:1247-55.
Hines, J. F., S. J. Ghim, N. D. Christensen, J. W. Kreider, W. A. Barnes, R. Schlegel, and A. B. Jenson. 1994. Role of conformational epitopes expressed by human papillomavirus major capsid proteins in the serologic detection of infection and prophylactic vaccination. Gynecol Oncol 55:13-20.
Jahan-Parwar, B., D. K. Chhetri, S. Hart, S. Bhuta, and G. S. Berke. 2003. Development of a canine model for recurrent respiratory papillomatosis. Ann Otol Rhinol Laryngol 112:1011-3.
Jarrett, W. F., et al., Studies on vaccination against papillomaviruses: prophylactic and therapeutic vaccination with recombinant structural proteins. Virology, 1991. 184(1): p. 33-42.
Jenson A B, Rosenthal J D, Olson C, Pass F, Lancaster W D, Shah K. Immunologic relatedness of papillomaviruses from different species. J Natl Cancer Inst 64:495-500, 1980.
Kawana, K., et al., A surface immunodeterminant of human papillomavirus type 16 minor capsid protein L2. Virology, 1998. 245(2): p. 353-9.
Kawana, K., et al., Common neutralization epitope in minor capsid protein L2 of human papillomavirus types 16 and 6. J Virol, 1999. 73(7): p. 6188-90.
Kawana, K., et al., In vitro construction of pseudovirions of human papillomavirus type 16: incorporation of plasmid DNA into reassembled L1/L2 capsids. J Virol, 1998. 72(12): p. 10298-300.
Kawana, K., et al., Nasal immunization of mice with peptide having a cross-neutralization epitope on minor capsid protein L2 of human papillomavirus type 16 elicit systemic and mucosal antibodies. Vaccine, 2001. 19(11-12): p. 1496-502.
Kawana, K., et al., Safety and immunogenicity of a peptide containing the cross-neutralization epitope of HPV16 L2 administered nasally in healthy volunteers. Vaccine, 2003. 21(27-30): p. 4256-60.
Kawana, Y., et al., Human papillomavirus type 16 minor capsid protein 12 N-terminal region containing a common neutralization epitope binds to the cell surface and enters the cytoplasm. J Virol, 2001. 75(5): p. 2331-6.
Kim, D., et al., Generation and characterization of a preventive and therapeutic HPV DNA vaccine. Vaccine, 2008. 26(3): p. 351-60.
Klinman et al., Proc. Natl. Acad. Sci., USA, 1996, 93, 2879-2883; WO98/16247
Knowles, G., et al., Linear B-cell epitopes in the N-terminus of L2 of bovine papillomavirus type 4. Res Vet Sci, 1997. 62(3): p. 289-91.
Koutsky L A, Ault K A, Wheeler C M, et al. A controlled trial of a human papillomavirus type 16 composition. N Engl J Med 2002; 347:1645-51.
Lacey C J, Thompson H S, Monteiro E F, et al. Phase IIa safety and immunogenicity of a therapeutic composition, TAGW, in persons with genital warts. J Infect Dis 1999; 179: 612-8.
Laemmli (1970) Cleavage of structural proteins during the assembly of the head of bacteriophage T4. Nature 227(5259):680-5.
Lin Y L, Borenstein L A, Selvakumar R, Ahmed R, Wettstein F O. Effective vaccination against papilloma development by immunization with L1 or L2 structural protein of cottontail rabbit papillomavirus. Virology 1992; 187:612-9.
Mao C, Koutsky L A, Ault K A, et al. Efficacy of human papillomavirus-16 composition to prevent cervical intraepithelial neoplasia: a randomized controlled trial. Obstet Gynecol 2006; 107:18-27.
Palmer, K. E., et al., Protection of rabbits against cutaneous papillomavirus infection using recombinant tobacco mosaic virus containing L2 capsid epitopes. Vaccine, 2006. 24(26): p. 5516-25.
Pastana D, Gambhira R, Buck C, Pang Y, Thompson C, Culp T, et al. Cross-neutralization of cutaneous and mucosal Papillomavirus types with anti-sera to the amino terminus of L2. Virology 2005; 337(2): 365-372.
Pastrana D V, Buck C B, Pang Y Y, et al. Reactivity of human sera in a sensitive, high-throughput pseudovirusbased papillomavirus neutralization assay for HPV16 and HPV18. Virology 2004; 321:205-16.
Pastrana D V, Buck C B, Pang Y Y, et al. Reactivity of human sera in a sensitive, high-throughput pseudovirusbased papillomavirus neutralization assay for HPV16 and HPV18. Virology 2004; 321:205-16.
Pastrana D V, Gambhira R, Buck C B, Pang Y Y, Thompson C D, Culp T D, Christensen N D, Lowy D R, Schiller J T, Roden R B. Cross-neutralization of cutaneous and mucosal Papillomavirus types with anti-sera to the amino terminus of L2. Virology. 337:365-72, 2005.
Pastrana, D. V., et al., Cross-neutralization of cutaneous and mucosal Papillomavirus types with anti-sera to the amino terminus of L2. Virology, 2005. 337(2): p. 365-72.
Richards, R M, D. R. Lowy, J. T. Schiller, and P. M. Day. (2006) Cleavage of the papillomavirus minor capsid protein, L2, at furin consensus site is necessary for infection. PNAS 103(5): 1522-1527.
Roden R B, Yutzy W, Fallon R, et al. Minor capsid protein of human genital papillomaviruses contains subdominant, cross-neutralizing epitopes. Virology 2000; 270:254-7.
Roden, R. B., et al., Minor capsid protein of human genital papillomaviruses contains subdominant, cross-neutralizing epitopes. Virology, 2000. 270(2): p. 254-7.
Shivprasad, S., G. P. Pogue, D. J. Lewandowski, J. Hidalgo, J. Donson, L. K. Grill, and W. O. Dawson. 1999. Heterologous sequences greatly affect foreign gene expression in tobacco mosaic virus-based vectors. Virology 255:312-23.
Slupetzky, K., et al., A papillomavirus-like particle (VLP) vaccine displaying HPV16 L2 epitopes induces cross-neutralizing antibodies to HPV11. Vaccine, 2007. 25(11): p. 2001-10.
Smith, M. L., Lindbo, J. A., Dillard-Telm, S., Brosio, P. M., Lasnik, A. B., McCormick, A. A., Nguyen, L. V., & Palmer, K. E. (2006) Modified tobacco mosaic virus particles as scaffolds for display of protein antigens for vaccine applications. Virology 348, 475-488.
Smyth U, Van Poelgeest M I, Davidson E J, et al. Immunological responses in women with human papillomavirus type 16 (HPV-16)-associated anogenital intraepithelial neoplasia induced by heterologous prime-boost HPV-16 oncogene vaccination. Clin Cancer Res 2004; 10:2954-61.
Suzich J A, Ghim S J, Palmer-Hill F J, White W I, Tamura J K, Bell J A, Newsome J A, Jenson A B, Schlegel R. Proc Natl Acad Sci USA. 1995 Dec. 5; 92(25):11553-7.
Towbin H, Staehelin T, Gordon J. Electrophoretic transfer of proteins from polyacrylamide gels to nitrocellulose sheets: procedure and some applications. Proc Natl Acad Sci USA. 1979 September; 76(9):4350-4.
van der Burg S H, Kwappenberg K M, O'Neill T, et al. Pre-clinical safety and efficacy of TA-CIN, a recombinant HPV16 L2E6E7 fusion protein composition, in homologous and heterologous prime-boost regimens. Composition 2001; 19:3652-60.
Vandepapeliere P, Barrasso R, Meijer C J, et al. Randomized controlled trial of an adjuvanted human papillomavirus (HPV) type 6 L2E7 composition: infection of external anogenital warts with multiple HPV types and failure of therapeutic vaccination. J Infect Dis 2005; 192: 2099-107.
Varsani, A., et al., Chimeric human papillomavirus type 16 (HPV-16) L1 particles presenting the common neutralizing epitope for the L2 minor capsid protein of HPV-6 and HPV-16. J Virol, 2003. 77(15): p. 8386-93.
Villa L L, Costa R L R, Petta C A, Andrade R P, Ault K A, Giuliano A R, et al. Prophylactic quadrivalent human papillomavirus (types 6, 11, 16, and 18) L1 virus-like particle composition in young women: a randomized double-blind placebo-controlled multicentre phase II efficacy trial. Lancet Oncol 2005; 6(5): 271-278.
Viscidi R P, Snyder B, Cu-Uvin S, et al. Human papillomavirus capsid antibody response to natural infection and risk of subsequent HPV infection in HIVpositive and HIV-negative women. Cancer Epidemiol Biomarkers Prev 2005; 14:283-8.
Yuan H, Ghim S, Newsome J, Apolinaro T, Olcese V, Martin M, Delius H, Felsburg P, Jenson B, Schlegel R. An epidermotropic canine papillomavirus with malignant potential contains an E5 gene and establishes a unique genus. Virology 2006; Oct. 9, 2006 electronic publication.
“Vaccine Design, The Subunit and Adjuvant Approach” published by M. Powell, M. Newman, Plenum Press 1995.
United States Patent Application Publication No. 2002/0197264 of Schlegel, et al.
United States Patent Application Publication No. 2003/0097683 of Lindbo, et al.
United States Patent Application Publication No. 2003/0108557 of Garger, et al.
United States Patent Application Publication No. 2004/0064855 of Pogue, et al.
United States Patent Application Publication No. 2004/0086527 of Schlegel, et al.
United States Patent Application Publication No. 2004/0088757 of Roberts, et al.
United States Patent Application Publication No. 2005/0009012 of Holzberg, et al.
United States Patent Application Publication No. 2005/0026257 of Gissmann, et al.
United States Patent Application Publication No. 2005/0175590 of Fitzmaurice, et al.
United States Patent Application Publication No. 2005/0282263 of McCormick, et al.
United States Patent Application Publication No. 2006/0029612 of Palmer, et al.
U.S. Pat. No. 2,909,462 to WARFIELD et al., issued Oct. 20, 1959, entitled “Acrylic acid polymer laxative compositions.”
U.S. Pat. No. 5,057,411 to Lancaster et al., issued Oct. 15, 1991, entitled “Type-specific papillomavirus DNA sequences and peptides.”
U.S. Pat. No. 5,283,171 to Manos et al., issued Feb. 1, 1994, entitled “Compositions for and detection of human papillomavirus by specific oligonucleotide polymerase primers using the polymerase chain reaction.”
U.S. Pat. No. 5,316,931 to Donson et al., issued May 31, 1994, entitled “Plant viral vectors having heterologous subgenomic promoters for systemic expression of foreign genes.”
U.S. Pat. No. 5,589,367 to Donson et al., issued Dec. 31, 1996, entitled “Recombinant plant viral nucleic acids.”
U.S. Pat. No. 5,639,606 to Willey, issued Jun. 17, 1997, entitled “Method for quantitative measurement of gene expression using multiplex competitive reverse transcriptase-polymerase chain reaction.”
U.S. Pat. No. 5,643,765 to Willey, issued Jul. 1, 1997, entitled “Method for quantitative measurement of gene expression using multiplex competitive reverse transcriptase-polymerase chain reaction.”
U.S. Pat. No. 5,811,653 to Turpen, issued Sep. 22, 1998, entitled “Viral amplification of recombinant messenger RNA in transgenic plants.”
U.S. Pat. No. 5,866,785 to Donson et al., issued Feb. 2, 1999, entitled “Recombinant plant viral nucleic acids.”
U.S. Pat. No. 5,874,089 to Schlegel et al., issued Feb. 23, 1999, entitled “Protecting against canine oral papillomavirus.”
U.S. Pat. No. 5,889,190 to Donson et al., issued Mar. 30, 1999, entitled “Recombinant plant viral nucleic acids.”
U.S. Pat. No. 5,889,191 to Turpen, issued Mar. 30, 1999, entitled “Viral amplification of recombinant messenger RNA in transgenic plants.”
U.S. Pat. No. 5,922,602 to Kumagai et al., issued Jul. 13, 1999, entitled “Cytoplasmic inhibition of gene expression.”
U.S. Pat. No. 5,965,794 to Turpen, issued Oct. 12, 1999, entitled “Viral amplification of recombinant messenger RNA in transgenic plants.”
U.S. Pat. No. 5,977,438 to Turpen et al., issued Nov. 2, 1999, entitled “Production of peptides in plants as viral coat protein fusions.”
U.S. Pat. No. 6,153,201 to Rose et al., issued Nov. 28, 2000, entitled “Oral immunization with papillomavirus virus-like particles.”
U.S. Pat. No. 6,165,471 to Garcea et al., issued Dec. 26, 2000, entitled “Homogeneous human papillomavirus capsomere containing compositions, methods for manufacture, and use thereof as diagnostic, prophylactic or therapeutic agents.”
U.S. Pat. No. 6,300,133 to Lindbo et al., issued Oct. 9, 2001, entitled “RNA transformation vectors derived from an uncapped single-component RNA virus.”
U.S. Pat. No. 6,300,134 to Lindbo et al., issued Oct. 9, 2001, entitled “RNA transformation vectors derived from a single-component RNA virus and contain an intervening sequence between the cap and the 5′ end.”
U.S. Pat. No. 6,376,752 to Kumagai et al., issued Apr. 23, 2002, entitled “Cytoplasmic inhibition of gene expression in a plant.”
U.S. Pat. No. 6,462,255 to Turpen, issued Oct. 8, 2002, entitled “Viral amplification of recombinant messenger RNA in transgenic plants.”
U.S. Pat. No. 6,479,291 to Kumagai et al., issued Nov. 12, 2002, entitled “Cytoplasmic inhibition of gene expression by viral RNA.”
U.S. Pat. No. 6,485,728 to Schlegel et al., issued Nov. 26, 2002, entitled “Formalin-Inactivated human papillomavirus L1 protein vaccine.”
U.S. Pat. No. 6,656,726 to Fitzmaurice et al., issued Dec. 2, 2003, entitled “Viral expression vectors.”
U.S. Pat. No. 6,660,500 to Turpen et al., issued Dec. 9, 2003, entitled “Production of peptides in plants as viral coat protein fusions.”
U.S. Pat. No. 6,700,040 to Roberts et al., issued Mar. 2, 2004, entitled “Cytoplasmic gene inhibition or gene expression in transfected plants by a tobraviral vector.”
U.S. Pat. No. 6,720,183 to Kumagai et al., issued Apr. 13, 2004, entitled “Cytoplasmic inhibition of gene expression.”
U.S. Pat. No. 6,800,748 to Holzberg et al., issued Oct. 5, 2004, entitled “Cytoplasmic inhibition of gene expression and expression of a foreign protein in a monocot plant by a plant viral vector.”
U.S. Pat. No. 6,887,478 to Schlegel et al., issued May 3, 2005, entitled “Formalin-treated human papillomavirus L1 protein vaccine.”
U.S. Pat. No. 6,908,615 to Hofmann et al., issued Jun. 21, 2005, entitled “DNA encoding human papilloma virus type 18.”
U.S. Pat. No. 7,001,995 to Neeper et al., issued Feb. 21, 2006, entitled “Synthetic human papillomavirus genes.”
U.S. Pat. No. 5,866,785 to Donson et al., issued Feb. 2, 1999, entitled “Recombinant plant viral nucleic acids”

What is claimed is:

1. A composition for treating a papillomavirus (PV) infection in a subject susceptible to PV infection, comprising: a PV L2 polypeptide produced from a eukaryotic expression system.

2. The composition of claim 1, wherein the PV L2 polypeptide comprises a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of a PV minor capsid (L2) protein.

3. The composition of claim 1, wherein the PV L2 polypeptide comprises a fragment selected from: 5-260, 9-150, 11-130, 13-70, 13-90, 13-120, 13-150, 13-180, and 13-200 of a PV minor capsid (L2) protein.

4. The composition of claim 1, wherein the PV L2 polypeptide comprises a fragment extending from a furin cleavage site to a downstream amino acid between about 65 and about 260 of a PV minor capsid (L2) protein.

5. The composition of claim 1, wherein the PV L2 polypeptide is a COPV L2 polypeptide.

6. The composition of claim 1, wherein the PV L2 polypeptide is an HPV L2 polypeptide.

7. The composition of claim 6, wherein the HPV L2 polypeptide is from an HPV-type, selected from the group consisting of: HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82.

8. The composition of claim 6, wherein the HPV L2 polypeptide is from a first HPV-type, and the composition is effective for treatment of infections caused by the first HPV-type and at least one additional HPV-type.

9. The composition of claim 8, wherein the first HPV-type is selected from an HPV-type of genus alpha-papillomavirus, and the composition is effective for treatment of the first HPV-type and at least one additional HPV-type of genus alpha-papillomavirus.

10. The composition of claim 9, wherein the first HPV-type is selected from an HPV-type of alpha-papillomavirus species 9, and the composition is effective for treatment of the first HPV-type and at least one additional HPV-type of alpha-papillomavirus species 9.

11. The composition of claim 10, wherein the first HPV-type is selected from HPV-16, 31, 33, 35, 52, 58, and 67, and the composition is effective for treatment of the first HPV-type and at least one additional HPV-type of alpha-papillomavirus species 9.

12. The composition of claim 9, wherein the first HPV-type is selected from an HPV-type of alpha-papillomavirus species 7, and the composition is effective for treatment of the first HPV-type and at least one additional HPV-type of alpha-papillomavirus species 7.

13. The composition of claim 12, wherein the first HPV-type is selected from HPV-18, 45, 49, 68, and 70, and the composition is effective for treatment of the first HPV-type and at least one additional HPV-type of alpha-papillomavirus species 7.

14. The composition of claim 9, wherein the first HPV-type is selected from an HPV-type of alpha-papillomavirus species 10, and the composition is effective for treatment of the first HPV-type and at least one additional HPV-type of alpha-papillomavirus species 10.

15. The composition of claim 14, wherein the first HPV-type is selected from HPV-6, 11, and 13, and the composition is effective for treatment of the first HPV-type and at least one additional HPV-type of alpha-papillomavirus species 10.

16. The composition of claim 6, wherein the composition is a multi-valent composition, comprising:
a first HPV L2 polypeptide from an HPV-type, selected from:
an HPV-type of alpha-papillomavirus species 9;
an HPV-type of alpha-papillomavirus species 7; and
an HPV-type of alpha-papillomavirus species 10; and
a second HPV L2 polypeptide from an HPV-Type, selected from:
an HPV-type of alpha-papillomavirus species 9;
an HPV-type of alpha-papillomavirus species 7; and
an HPV-type of alpha-papillomavirus species 10; and
wherein the first and second HPV L2 polypeptides are selected from different species.

17. The composition of claim 16, wherein the composition is a multi-valent composition, further comprising:
a third HPV L2 polypeptide from an HPV-type, selected from:
an HPV-type of alpha-papillomavirus species 9;
an HPV-type of alpha-papillomavirus species 7;
an HPV-type of alpha-papillomavirus species 10; and
wherein the first, second, and third HPV L2 polypeptides are selected from different species.

18. The composition of claim 17, wherein the composition is effective for treatment of the HPV-types of alpha-papillomavirus species 9, the HPV-types of alpha-papillomavirus species 7, and the HPV-types of alpha-papillomavirus species 10.

19. The composition of claim 18, wherein the first HPV L2 polypeptide is from an HPV-type, selected from: HPV-16, 31, 33, 35, 52, 58, and 67; the second HPV L2 polypeptide is from an HPV-type, selected from: HPV-18, 45, 49, 68, and 70; and the third HPV L2 polypeptide is from an HPV-type, selected from: HPV-6, 11, and 13.

20. The composition of claim 19, wherein the first HPV L2 polypeptide is from HPV-16; the second HPV L2 polypeptide is from HPV-18; and the third HPV L2 polypeptide is from HPV-6.

21. The composition of claim 1, wherein the composition comprises a fusion protein comprising the PV L2 polypeptide.

22. The composition of claim 21, wherein the fusion protein further comprises a streptavidin (SA).

23. The composition of claim 1, wherein the PV L2 polypeptide is conjugated to a tobacco mosaic virus (TMV).

24. The composition of claim 1, wherein the eukaryotic expression system is a plant-based expression system.

25. The composition of claim 24, wherein the plant-based expression system comprises a tobacco mosaic virus (TMV)-based DNA plasmid.

26. The composition of claim 1, further comprising a pharmaceutically-acceptable carrier.

27. The composition of claim 1, further comprising an adjuvant.

28. The composition of claim 1, wherein treating the PV infection prevents or reduces the risk of PV infection.

29. The composition of claim 1, wherein the composition for treating PV infection elicits an immune response in the subject.

30. The composition of claim 1, wherein the composition for treating PV infection prevents, reduces the risk of, ameliorates, or relieves symptoms of PV infection.

31. The composition of claim 30, wherein the PV infection is an HPV infection in a human subject and the symptoms include formation of papillomas or warts, development of precancerous lesions, development of cancer, and combinations thereof.

32. A method of treating human papillomavirus (HPV) infection by administering an effective amount of the composition of claim 1, wherein the PV L2 polypeptide is an HPV L2 polypeptide.

33. The method of claim 32, wherein administering the effective amount of the composition immunizes against the HPV infection.

34. A method of treating canine oral papillomavirus (COPV) infection by administering an effective amount of the composition of claim 1, wherein the PV L2 polypeptide is a COPV L2 polypeptide.

35. The method of claim 34, wherein administering the effective amount of the composition immunizes against the COPV infection.

36. A method of producing a PV L2 polypeptide in a eukaryotic expression system, comprising:
identifying a PV L2 polypeptide of interest;
generating an expression vector comprising a gene encoding the HPV L2 polypeptide;
transcribing the gene;
introducing the transcribed gene into at least one eukaryotic cell;
expressing the PV L2 polypeptide from the transcribed gene within the eukaryotic cell; and
isolating the PV L2 polypeptide from the eukaryotic cell.

37. The method of claim 36, wherein the expression vector is a tobacco mosaic virus (TMV)-based DNA plasmid.

38. The method of claim 36, wherein the gene is under the control of a regulatory element.

39. The method of claim 38, wherein the regulatory element is a T7 promoter.

40. The method of claim 39, wherein the transcribing comprises in vitro transcription using T7 polymerase.

41. The method of claim 36, wherein the introducing comprises infecting the eukaryotic cell with the transcribed gene.

42. The method of claim 36, wherein the eukaryotic cell is a Nicotiana spp. cell.

43. The method of claim 42, wherein the Nicotiana spp. cell is a plurality of cells comprising a Nicotiana spp. seedling.

44. The method of claim 36, wherein the isolating comprises lysing the eukaryotic cell and purifying the HPV L2 polypeptide from the lysed cell.

45. The method of claim 36, wherein the PV L2 polypeptide comprises a fragment extending from amino acid 5, 6, 7, 8, 9, 10, 11, 12, 13, 14, 15, 16, 17, 18, 19, 20, 21, 22, 23, 24, or 25, and extending to amino acid 150, 145, 140, 135, 130, 125, 120, 115, 110, 105, 100, 95, 90, 85, 80, 75, 74, 73, 72, 71, 70, 69, 68, 67, 66, or 65 of COPV minor capsid (L2) protein.

46. The method of claim 36, wherein the PV L2 polypeptide comprises a fragment selected from: 5-260, 9-150, 11-130, 13-70, 13-90, 13-120, 13-150, 13-180, and 13-200.

47. The method of claim 36, wherein the composition comprises a fusion protein comprising the PV L2 polypeptide.

48. The method of claim 47, wherein the fusion protein further comprises a streptavidin (SA).

49. The method of claim 36, wherein the PV L2 polypeptide is conjugated to a tobacco mosaic virus (TMV).

50. The method of claim 36, wherein the PV L2 polypeptide comprises a fragment extending from a furin cleavage site to a downstream amino acid between about 65 and about 260 of a PV minor capsid (L2) protein.

51. The method of claim 36, wherein the PV L2 polypeptide is a COPV L2 polypeptide.

52. The method of claim 36, wherein the PV L2 polypeptide is a HPV L2 polypeptide.

53. The method of claim 52, wherein the HPV L2 polypeptide is from an HPV-type, selected from the group consisting of: HPV-6, HPV-11, HPV-16, HPV-18, HPV-26, HPV-31, HPV-33, HPV-35, HPV-39, HPV-40, HPV-45, HPV-51, HPV-52, HPV-53, HPV-56, HPV-58, HPV-59, HPV-68, HPV-73, and HPV-82.

54. The method of claim 52, wherein the HPV L2 polypeptide is from a first HPV-type, and the composition is effective for treatment of infections caused by the first HPV-type and at least one additional HPV-type.

55. The method of claim 54, wherein the first HPV-Type is selected from an HPV-type of genus alpha-papillomavirus, and the composition is effective for treatment of the first HPV-type and at least one additional HPV-Type of genus alpha-papillomavirus.

56. The method of claim 55, wherein the first HPV-Type is selected from an HPV-type of alpha-papillomavirus species 9, and the composition is effective for treatment of the first HPV-type and at least one additional HPV-type of alpha-papillomavirus species 9.

57. The method of claim 56, wherein the first HPV-type is selected from HPV-16, 31, 33, 35, 52, 58, and 67, and the composition is effective for treatment of the first HPV-type and at least one additional HPV-type of alpha-papillomavirus species 9.

58. The method of claim 55, wherein the first HPV-type is selected from an HPV-type of alpha-papillomavirus species 7, and the composition is effective for treatment of the first HPV-type and at least one additional HPV-type of alpha-papillomavirus species 7.

59. The method of claim 58, wherein the first HPV-type is selected from HPV-18, 45, 49, 68, 70, and the composition is effective for treatment of the first HPV-type and at least one additional HPV-type of alpha-papillomavirus species 7.

60. The method of claim 55, wherein the first HPV-type is selected from an HPV-type of alpha-papillomavirus species 10, and the composition is effective for treatment of the first HPV-type and at least one additional HPV-type of alpha-papillomavirus species 10.

61. The method of claim 60, wherein the first HPV-type is selected from HPV-6, 11, and 13, and the composition is effective for treatment of the first HPV-type and at least one additional HPV-type of alpha-papillomavirus species 10.

62. The method of claim 52, wherein the composition is a multi-valent composition, comprising:
a first HPV L2 polypeptide from an HPV-Type, selected from:
an HPV-type of alpha-papillomavirus species 9;
an HPV-type of alpha-papillomavirus species 7; and
an HPV-type of alpha-papillomavirus species 10; and
a second HPV L2 polypeptide from an HPV-Type, selected from:
an HPV-type of alpha-papillomavirus species 9;
an HPV-type of alpha-papillomavirus species 7; and
an HPV-type of alpha-papillomavirus species 10; and
wherein the first and second HPV L2 polypeptides are selected from different species.

63. The method of claim 62, wherein the composition is a multi-valent composition, further comprising:
a third HPV L2 polypeptide from an HPV-Type, selected from:
an HPV-type of alpha-papillomavirus species 9;
an HPV-type of alpha-papillomavirus species 7; and
an HPV-type of alpha-papillomavirus species 10; and
wherein the first, second, and third HPV L2 polypeptides are selected from different species.

64. The method of claim 63, wherein the composition is effective for treatment of the HPV-types of alpha-papillomavirus species 9, the HPV-types of alpha-papillomavirus species 7, and the HPV-types of alpha-papillomavirus species 10.

65. The method of claim 64, wherein the first HPV L2 polypeptide is from an HPV-type, selected from: HPV-16, 31, 33, 35, 52, 58, and 67; the second HPV L2 polypeptide is from an HPV-type, selected from: HPV-18, 45, 49, 68, and 70; and the third HPV L2 polypeptide is from an HPV-type, selected from: HPV-6, 11, and 13.

66. The method of claim 65, wherein the first HPV L2 polypeptide is from HPV-16; the second HPV L2 polypeptide is from HPV-18; and the third HPV L2 polypeptide is from HPV-6.
